(19) United States
(12) Patent Application Publication
(10) Pub. No.: US 2003/0004149 A1
(43) Pub. Date: Jan. 2, 2003

(54) Antiangiogenic compounds and an assay for inhibitors of cell invasion

(76) Inventors: Calvin Roskelley, Vancouver (CA); Raymond Andersen, Vancouver (CA); David Williams, Vancouver (CA); Michel Roberge, Vancouver (CA); Shoukat Dedhar, Richmond (CA); Aly Karsan, Vancouver (CA); Andrew Minchinton, Vancouver (CA)

Correspondence Address: BOZICEVIC, FIELD & FRANCIS LLP, 200 MIDDLEFIELD RD, SUITE 200, MENLO PARK, CA, 94025, US

(21) Appl. No.: 10/057,846

(22) Filed: Jan. 25, 2002

Related U.S. Application Data
(60) Provisional application No. 60/330,670, filed on Oct. 26, 2001.

(30) Foreign Application Priority Data
Jan. 25, 2001 (CA) 2,332,138

(51) Int. Cl. A61K 31/33; C07D 225/00

(52) U.S. Cl. 514/183; 540/450

(57) ABSTRACT

This invention provides the use of macrocyclic amines for inhibition of cellular invasion or angiogenesis. Compounds and pharmaceutical compositions of this invention are useful in the treatment of conditions characterized by cellular invasion or angiogenesis, including cancer. Compounds that may be used in this invention include the motuporamines.

### FIELD OF INVENTION

This invention relates to assays for agents that affect cellular invasions and the use of agents that inhibit cellular invasions or angiogenesis in the treatment of disease.

### BACKGROUND OF INVENTION

Cell motility and invasion are essential physiological processes in tissue development and homeostasis, including embryogenesis, angiogenesis, wound healing, ovulation, embryo implantation and pregnancy, immune surveillance and inflammation. They are also key factors in many pathological processes such as inflammation, atherosclerosis, restenosis, glaucoma, retinopathies, myocardial ischemia, rheumatoid arthritis, psoriasis, and tumour progression and metastasis. For example, the process of tumour metastasis begins with the dissemination of cells from the primary tumour followed by the movement of cells through the stromal compartment of the organ in which the primary tumour is located, intravasation of the tumour cells from vascular/lymphatic bed within distant, secondary organ of metastasis, and the movement of tumour cells into the tissues of the secondary organ.

Inhibition of cell motility and invasion would be useful for the treatment of cancer, and other disorders involving cell motility and invasion including those listed above, as well as for contraception. For example, cancer cell invasion driven by altered interactions between cells and an extracellular matrix (ECM). In the case of epithelial-derived carcinomas, the primary tumour is surrounded by a specialized ECM, the basement membrane. Tissue culture procedures which utilize reconstituted basement membrane matrices have been used to demonstrate that changes in matrix deposition, matrix degradation, cellular attachment to the matrix and migration through the matrix play a role in carcinoma cell invasion (Wyke, J. A. (2000) Eur. J. Cancer, 36:1589-1594).

Previous methods for determining whether an agent has an effect on cellular invasion rely on the use of a biological matrix and involve detection of a change in cell penetration or migration through such a matrix. In particular, such methods have involved measuring a decrease in the number of cells (or the absence of cells) which penetrate or migrate trough a matrix upon application of an agent. Such methods are suitable for use in situations where information related to the agent's cytotoxicity or ability to affect a cell's attachment to the biological matrix is known, but are not generally suitable for use in screening procedures.

Angiogenesis inhibition presents a cancer treatment strategy because avascular tumours are incapable of growth and have little metastatic potential. Antiangiogenic compounds that have been evaluated for treatment of solid tumours include small molecules that inhibit metalloproteinases, ion channels, protein kinases, or cell proliferation; agents that inactivate or antagonize growth factors; and agents whose mechanism of action is still unknown. Discovery of new antiangiogenic agents is desirable for the development of therapies for cancer and other diseases.

### SUMMARY OF INVENTION

This invention provides the use of compounds or pharmaceutically acceptable salts of compounds of formula I or II as inhibitors of cellular invasion or as antiangiogenic agents, wherein formula I is:

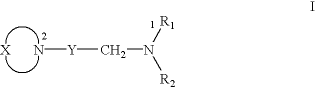

wherein:

X is a saturated, or unsaturated linear, branched, or partially cyclized alkyl chain of between eleven and thirty carbons that may be substituted with one or more epoxide, ketone (═O), thiocarbonyl (═S), oxime (═N—OH), —OH, —OR, —O₂CR, —SH, —SR, —SOCR, —NH₂, —NHR, —NR₂, —NR³+, —NHCOR, —I, —Br, —Cl, —F, —CN, —CO₂H, —CO₂R, —CHO, —COR, —CONH₂, —CONHR, NRCOR, —CONR₂, COSR, —NO₂, —OSO₃H, —SO₃H, —SOR, —SO₂R; wherein one or more CH₂ groups if present, in the alkyl chain may be replaced by O, S, NH, or NR; and wherein one or more C and CH groups if present, in the alkyl chain may be replaced with NH or NR;

R₁ and R₂ are independently: hydrogen; methyl; a linear, branched, or cyclic saturated, or unsaturated alkyl group containing one to ten carbons that may be substituted with one or more —OH, —OR, ═O, ═S, ═N—OH, —O₂CR, —SH, —SR, —SOCR, —NH₂, —NHR, —NR₂, —NR₃+, —NHCOR, —I, —Br, —Cl, —F, —CN, —CO₂H, —CO₂R, —CHO, —COR, CONH₂, —CONHR, NRCOR, —CONR₂, —COSH, —COSR, —CSOR, NO₂, —OSO₃H, —SO₃H, —SOR, —SO₂R; or benzyl wherein a phenyl ring of the benzyl may be substituted with one or more R, —OH, —OR, —O₂CR, —SH, —SR, —SOCR, —NH₂, —NHR, —NHR₂, —NHCOR, —I, —Br, —Cl, —F, —CN, —CO₂H, —CO₂R, —CHO, —COR, —CONH₂, —CONHR, —CONR₂, —COSH, —COSR, —NO₂, —SO₃H, —SO₂R; providing neither of R₁ and R₂ is an acyl or thioacyl residue forming an amide with N¹;

Y is a linear, branched, or cyclic, saturated, or unsaturated alkyl chain containing one to ten carbons that may be substituted with one or more epoxide —OH, —OR, ═O, ═S, ═N—OH, —O₂CR, —SH, SR, —I, —Br, —Cl, —F, —CN, —CO₂R, —CHO, —COR, —CONH₂, —CONHR, NRCOR, —CONR₂, NO₂, —SOR, —SO₂R; wherein one or more CH₂ groups if present, in the alkyl chain may be replaced by O or S;

R is defined as: a linear, branched, or cyclic one to ten carbon saturated, or unsaturated alkyl group that may be substituted with one or more epoxide —OH, —OR′, ═O, ═S, ═N—OH, —O₂CR′; —SH, —SR′, —SOCR′, —OSO₃H, —NH₂, —NHR′, —NHR′₂, —NR₃+, —NHCOR′, NR′COR′, —I, —Br, —Cl, —F, —CN, —CO₂H, —CO₂R′, —CHO, —COR′, CONH₂, —CONHR′, —CONR′₂, —COSH, —COSR′, —NO₂, —SO₃H, —SOR′, —SO₂R′; wherein R′ is a linear, branched, or cyclic one to ten carbon, saturated, or unsaturated alkyl group that may be substituted with —NH₂;

and wherein formula II is:

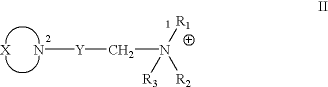

wherein:

X is a saturated, or unsaturated linear, branched, or partially cyclized chain of between eleven and thirty carbons that may be substituted with one or more epoxide, ketone (═O), thiocarbonyl (═S), oxime (═N—OH), —OH, —OR, —O₂CR, —SH, —SR, —SOCR, —NH₂, —NHR, —NR₂, —NR₃+, —NHCOR, —I, —Br, —Cl, —F, —CN, —CO₂H, —CO₂R, —CHO, —COR, —CONH₂, —CONHR, NRCOR, —CONR₂, —COSR, —NO₂, —OSO₃H, —SO₃H, —SOR, —SO₂R; wherein one or more CH₂ groups in the alkyl chain if present, may be replaced by O, S, NH, or NR; and wherein one or more C or CH groups in the alkyl chain if present, may be replaced with NH or NR;

R₁, R₂, and R₃ are independently: methyl; a linear branched, or cyclic, saturated, or unsaturated alkyl group containing one to ten carbons that may be substituted with one or more —OH, —OR, ═O, ═S, ═N—OH, —O₂CR, —SH, —SR, —SOCR, —NH₂, —NHR, —NR₂, —NR₃+, —NHCOR, —I, —Br, —Cl, —F, —CN, —CO₂H, —CO₂R, —CHO, —COR, —CONH₂, —CONHR, NRCOR, —CONR₂, —COSH, —COSR, —CSOR, NO₂, OSO₃H, —SO₃H, —SOR, —SO₂R; or benzyl wherein a phenyl ring of the benzyl may be substituted with one or more R, —OH, OR, —O₂CR, —SH, —SR, —SOCR, —NH₂, —NHR, —NHR₂, —NHCOR, —I, —Br, —Cl, —F, —CN, —CO₂H, —CO₂R, —CHO, —COR, —CONH₂, —CONHR, CONR₂, —COSH, —COSR, —NO₂, —SO₃H, —SOR, —SO₂R; and, providing none of R₁, R₂, and R₃ is an acyl or thioacyl residue forming an amide with N¹;

Y is a linear, branched, or cyclic, saturated, or unsaturated alkyl chain containing one to ten carbons that may be substituted with one or more epoxide —OH, —OR, ═O, ═S, ═N—OH, —O₂CR, —SH, —SR, —I, —Br, —Cl, —F, —CN, —CO₂R, —CHO, —COR, —CONH₂, —CONHR, NRCOR, —CONR₂, NO₂, —SOR, —SO₂R; wherein one or more CH₂ groups if present, in the alkyl chain may be replaced by O or S; and,

R is defined as: a linear, branched, or cyclic one to ten carbon saturated, or unsaturated alkyl group that may be substituted with one or more epoxide, —OH, —OR′, ═O, ═S, ═N—OH, —O₂CR′, —SH, —SR′, —SOCR′, —OSO₃H, —NH₂, —NHR′, —NHR′₂, —NR′₃+, —NHCOR′, NR′COR′, —I, —Br, —Cl, —F, —CN, —CO₂H, —CO₂R′, —CHO, —COR′, —CONH₂, —CONHR′, —CONR′₂, —COSH, —COSR′, —NO₂, —SO₃H, —SOR′, —SO₂R′, wherein R′ is a linear, branched, or cyclic one to ten carbon, saturated, or unsaturated alkyl group that may be substituted with —NH₂.

Preferably, compounds of formula I or II will be a compound having one or more of the following:

(a) Y is (CH₂)ₙ, wherein n is 1-5, optionally substituted as described above;

(b) X is a saturated linear or branched alkyl chain of 11-16 carbon atoms, optionally substituted with R;

(c) X is an unsaturated linear or branched alkyl chain of 11-16 carbon atoms, optionally substituted with R;

(d) X is a fully unsaturated and partially cyclized linear alkyl chain of 11-16 carbon atoms optionally substituted with R;

(e) for formula I, one of R₁ and R₂ is a linear or branched alkyl group optionally substituted with NH₂, —NHR, —NR₂, —NR₃+, or —NHCOR;

(f) for formula I, one of R₁ and R₂ is hydrogen; methyl; or, a linear or branched alkyl group optionally substituted with —OH, —OR, or ═O;

(g) for formula I, one of R₁ and R₂ is a linear or branched C₂ to C₆ alkyl group optionally substituted with NH₂, —NHR, —NR₂, —NR₃+, or —NHCOR,

(h) for formula I, one of R₁ and R₂ is hydrogen; methyl; or, a linear or branched C₂ to C₆ alkyl group optionally substituted with —OH, —OR, or ═O;

(i) for formula II, one of R₁, R₂, and R₃ is a linear or branched alkyl group optionally substituted with NH₂, —NHR, —NR₂, —NR₃+, or —NHCOR;

(j) for formula II, one of R₁, R₂, and R₃ is methyl; or, a linear or branched alkyl group optionally substituted with —OH, —OR, or ═O.

(k) for formula II, one of R₁, R₂, and R₃ is a linear or branched C₂ to C₆ alkyl group optionally substituted with NH₂, —NHR, —NR₂, —NR₃+, or —NHCOR; and,

(l) for formula II, one of R₁, R₂, and R₃ is methyl; or, a linear or branched C₂ to C₆ alkyl group optionally substituted with —OH, —OR, or ═O.

Compounds of formula I may be one in which:

(a) Y is (CH₂)ₙ wherein n is 1-5, more preferably n is 1, 2, or 3;

(b) X is alternatively: a saturated or unsaturated linear alkyl chain of 11-15 carbon atoms, optionally substituted with R, with R preferably being a C₁-C₆ linear or branched alkyl group; or, X is a fully unsaturated and partially cyclized linear alkyl chain of 11-16 carbon atoms;

(c) one of R₁ and R₂ is H, methyl, or a linear or branched C₂-C₆ alkyl group; and,

(d) another of R₁ and R₂ is a linear or branched C₂-C₆ alkyl group optionally substituted with NH₂, —NHR, —NR₃+, or —NHCOR, wherein R is a linear or branched C₁-C₆ saturated or unsaturated alkyl group.

Compounds of formula II may be one in which:

(a) Y is (CH₂)ₙ, wherein n is 1 -5, more preferably n is 1, 2, or 3;

(b) X is alternatively: a saturated or unsaturated linear alkyl chain of 11-15 carbon atoms, optionally substituted with R, with R preferably being a C₁-C₆ linear or branched alkyl group; or, X is a fully unsaturated and partially cyclized linear alkyl chain of 11-16 carbon atoms;

(c) one or two, and more preferably two of R₁, R, and R₃ is/are a methyl or a linear or branched C₂-C₆ alkyl group; and,

(d) one of R₁, R, and R₃ is a linear or branched C₂-C₆ alkyl group optionally substituted with NH₂, —NHR, NR₃+, or —NHCOR, wherein R is a linear or branched C₁-C₆ saturated or unsaturated alkyl group.

The terms “unsaturated” and more particularly “fully unsaturated”, as used herein, include aromatic structures.

The term “partially cyclized” as used herein with respect to the alkyl chain “X” means that the chain includes one or more ring structures. Thus, for either formula I or II, the structure formed by “X” and “N²” may be a single ring or where “X” is partially cyclized, the structure will comprise multiple rings.

This invention provides compounds of formula I or II, and pharmaceutical compositions comprising an acceptable carrier and one or more compounds of formula I or II, for treatment or for contraception. This invention also provides the use of such compounds or salts, for the preparation of medicaments for treatment of disease conditions, for inhibition of cellular invasion, motility, or angiogenesis or for contraception. This invention also provides the use of such compounds, salts, pharmaceutical compositions, and medicaments for the treatment of disease conditions, for inhibition of cellular invasion, motility or angiogenesis and for contraception. Representative disease conditions include cancer, inflammation, atherosclerosis, restenosis, arthritis, psoriasis, glaucoma, retinopathies, and myocardial ischemia. Other conditions or circumstances where treatment to inhibit cellular invasion or motility may be done in accordance with this invention is to reduce axonal plasticity during neuronal recovery (for example, during treatment following neuronal damage such as a neural or spinal cord lesion). This invention also provides methods for treatment of such conditions, methods for inhibiting cellular invasion or motility, and methods of contraception, comprising administration of a compound, a pharmaceutical salt of a compound, or a pharmaceutical preparation comprising one or more such compounds or salts thereof, to a subject in need of or desiring such treatment, wherein the compound is an inhibitor of cellular invasion, motility or angiogenesis and is a compound of formula I or II.

This invention also provides novel compounds of formula I or II; novel salts of compounds of formula I or II; and, pharmaceutical preparations comprising an acceptable carrier and a compound of formula I or II or salt thereof. Compounds of formula I or II which are not novel are Motuporamines A, B, and C as described in Williams, D. E., et al. (1998) J. Org. Chem. 63:4838-4841. However, Motuporamines A, B, and C are included in the pharmaceutical preparations, uses, and methods of this invention. These known motuporamines are mildly cytotoxic macrocyclic alkaloids initially isolated from a tropical sponge of the species Xestospongia exigua (Williams, et al. [supra]).

Various methods for synthesis of the previously known motuporamines are known and have been reported in the literature (see: Goldring, W. P. D. and Weiler, L. (1999) Organic Letters 1:1471-1473. Goldring, W.P.D. et al. (1998) Tetrahedron Letters 39:4955-5948; and, Furstner, A. and Rumbo, A. (2000) J. Org. Chem. 65:2608-2611; and, Baldwin, J. E., et al. (1999) Tetrahedron Letters 40:5401-5404). Compounds and pharmaceuticals salts thereof for use in this invention, including the novel motuporamines and salts described herein may be obtained from natural sources and subsequently modified as disclosed in the latter references and in the examples herein. Preferably, compounds for use in this invention will be synthesized, for example by using a known synthetic route, with appropriate modification to starting materials and procedures as would be apparent to a person of skill in the art, to obtain novel motuporamines as described herein. Prior methods for modification of the known motuporamines, and the specific methodologies for producing analogs described herein, may be used or adapted by a person of skill in the art for preparation of novel motuporamines and analogs as covered by this invention.

This invention also provides a method for testing for the presence of an agent that inhibits cellular invasion comprising:

(a) placing invasive cells on a surface of a biological matrix;

(b) treating said cells with an agent to be tested for cellular invasion inhibition activity;

(c) maintaining the cells on the surface of the matrix for a time sufficient for the cells to invade the matrix;

(d) removing substantially all cells from the surface of the matrix after (c);

(e) transferring the cells removed at (d) to a surface upon which said cells are capable of attachment and proliferation;

(f) maintaining the cells on a surface at (e) for a time sufficient for cellular attachment and proliferation on the surface; and

(g) determining a value indicative of a quantity of cells attached to said surface after (f).

In the preceding method, (a) and (b) may occur simultaneously or in any order. The cells are capable of invading the matrix and the matrix is such that the cells will exhibit invasiveness in absence of an inhibitor of cellular invasion.

The above-described testing method may also comprise comparing the value determined at (g) to a value determined for a control. The control may be cells for which the method is performed under the same conditions except that treatment of the cells at (b) is not performed in tee control. In the latter case, the method of this invention may additionally comprise comparing the value at (g) for the agent to the value at (g) for a control, to provide a value indicative of the amount of inhibition by the agent. A result indicative of the agent being an inhibitor of cellular invasion is one in which the value determined at (g) in the method of this invention is less than the value determined for a control.

### BRIEF DESCRIPTION OF DRAWINGS

FIG. 1: is a graph comparing percent invasion inhibition using the assay of this invention to varying amounts of Motuporamine A. The structure of Motuporamine A is also shown.

FIG. 2: is a graph comparing percent invasion inhibition using the assay of this invention to varying amounts of a mixture of Motuporamine B and D. The structures of Motuporamine B and D are also shown.

FIG. 3: is a graph comparing percent invasion inhibition using the assay of this invention to varying amounts of Motuporamine C. The structure of Motuporamine C is also shown.

FIG. 4: is a graph comparing percent invasion inhibition using the assay of this invention to varying amounts of Motuporamine E. The structure of Motuporamine E is also shown.

FIG. 5: is a graph comparing percent invasion inhibition using the assay of this invention to varying amounts of monoacetylated Motuporamine C. The structure of monoacetylated Motuporamine C is also shown.

FIG. 6: is a graph comparing percent invasion inhibition using the assay of this invention to varying amounts of diacetylated Motuporamine C after acid hydrolysis. The structure of diacetylated Motuporamine C after hydrolysis is also shown.

FIG. 7: is a graph comparing percent invasion inhibition using the assay of this invention to varying amounts of a compound having a motuporamine ring structure but lacking a spermidine-like tail. The structure of the compound is also shown.

FIG. 8: is a graph comparing percent invasion inhibition using the assay of this invention to varying amounts of CF₃Ac-Motuporamine C. The structure of CF₃Ac-Motuporamine C is also shown.

FIG. 9: is a graph comparing percent invasion inhibition using the assay of this invention to varying amounts of a motuporamine analog of this invention. The structure of the analog is also shown.

FIG. 10: is a graph comparing percent invasion inhibition using the assay of this invention to varying amounts of a motuporamine analog. The structure of the analog is also shown. FIG. 11: is a graph comparing percent invasion inhibition using the assay of this invention to varying amounts of a motuporamine analog of this invention. The structure of the analog is also shown.

FIG. 12: is a chart comparing percent invasion inhibition using the assay of this invention to varying amounts of motuporamine carbazole analog of this invention. The structure of the analog is also shown.

FIG. 13: is a chart showing the structure of other motuporamine analogs of this invention which are inhibitors of cell invasion.

FIG. 14: shows the generic structure of a methylated form of Motuporamine A (FIG. 14A), which includes Motuporamines G, H, and I each having a methyl substituted at one of C-12 to C-15 of Motuporamine A (FIG. 14B).

FIG. 15: shows the structure of Dihydroymotuporamine C.

### DETAILED DESCRIPTION OF THE INVENTION

In the testing method of this invention, invasive cells are placed on the surface of a biological matrix. For purposes of this invention, invasive cells are any cells able to move through a biological membrane or a gel made of extracellular matrix materials or other materials compatible with cell survival. Suitable cells include any invasive cancer cells such as those described in the examples below or other cells such as PC-3 prostate carcinoma, U-87 glioma, and U-251 glioma, as well as non-cancerous cells such as neuronal cells, endothelial cells, nucleated hemopoietic cells, smooth muscle cells, and fibroblasts.

For purposes of this invention, a biological matrix may be a membrane or a gel made of extracellular matrix materials or other materials compatible with cell survival into which invasive cells are capable of invading. Examples include the biological matrix employed in the examples below as well as other gels such as fibrin gels, or gels formed or individual extracellular matrix components or mixtures of extracellular components, or gels formed of any substance that is compatible with cell survival, or gels containing any substances that are compatible with cell survival. Suitable biological matrices include those used to date to assess the invasive qualities of cells.

In the testing method of this invention, invasive cells are maintained on the surface of a biological matrix for a time sufficient for the cells to invade the matrix. The time may be determined by the person of skill in the art based on the type of cells employed and the nature of the matrix. Once sufficient time has elapsed for the cells to invade the matrix, substantially all cells are removed from the surface of the matrix. This may be done using any suitable means known in the art, including those described in the examples below. The removed cells are transferred to a surface upon which the cells are capable of attachment and proliferation. Such a surface may be any surface known in the art that is compatible with the cells employed in the testing method and include the plastic surfaces described herein. The cells are maintained on the surface for a time sufficient for the cells to attach and proliferate on the surface. The amount of time will be selected by the practitioner according to the type of cell and nature of the surface in order that a sufficient amount of time will lapse such that cells that have attached to the surface and are alive will have proliferated.

In the testing method of this invention, a value is determined indicative of the number or quantity of cells that are attached to the surface and have proliferated. Various means known in the art may be used for determining this value including means for directly counting the number of cells, as well as indirect means such as the assay procedures described herein. Other procedures include the MTS, neutral red, radioactive thymidine incorporation assays as well as others capable of measuring the quantity or numbers of live cells.

Compounds of this invention or for use in this invention, including pharmaceutically acceptable salts thereof, may be obtained by synthesis according to the previously known procedures as described above or as exemplified herein. Some compounds that may be used in this invention can be obtained from natural sources, such as is disclosed in the prior art and in the examples herein. Such naturally occurring motuporamines may be modified according to methodology known in the art and as specifically exemplified herein. Compounds and pharmaceutically acceptable salts thereof of this invention and for use in this invention will be in substantially purified form. By “substantially purified formula”, it is meant that the compound or salt (if naturally occurring), is present substantially free of cells of a source organism or tissue. The term “substantially free of cellular contaminants” means that the compound is present ex vivo and in a concentration greater than that of the compound in a source organism, tissue, or other natural source.

The synthesis schemes shown in Table 1 generally outline reactions that may be used to prepare motuporamine compounds and analogs thereof for use in this invention. Examples of use of such schemes for preparation of specific compounds is described in the examples which follow.

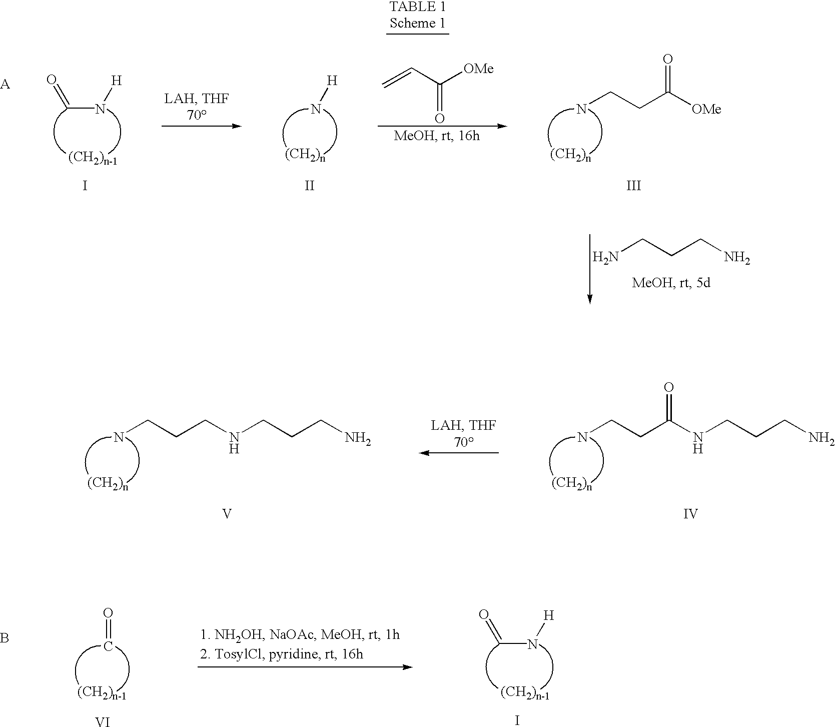

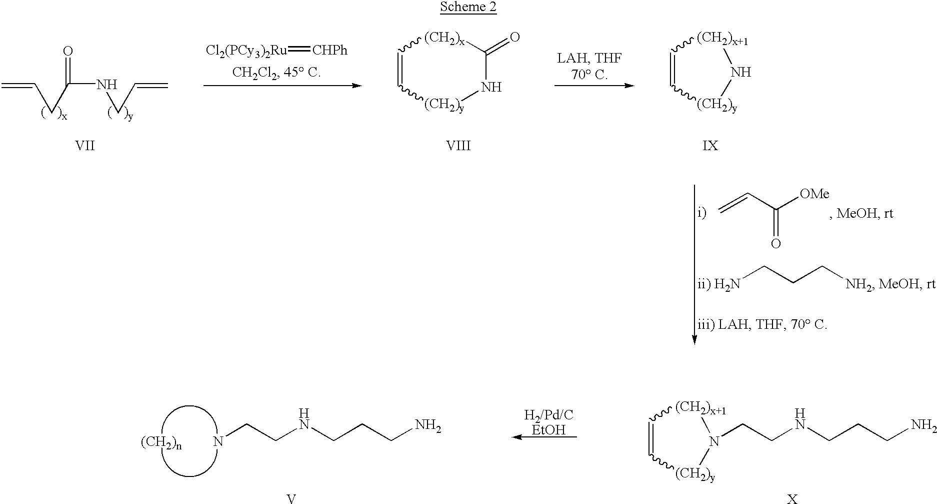

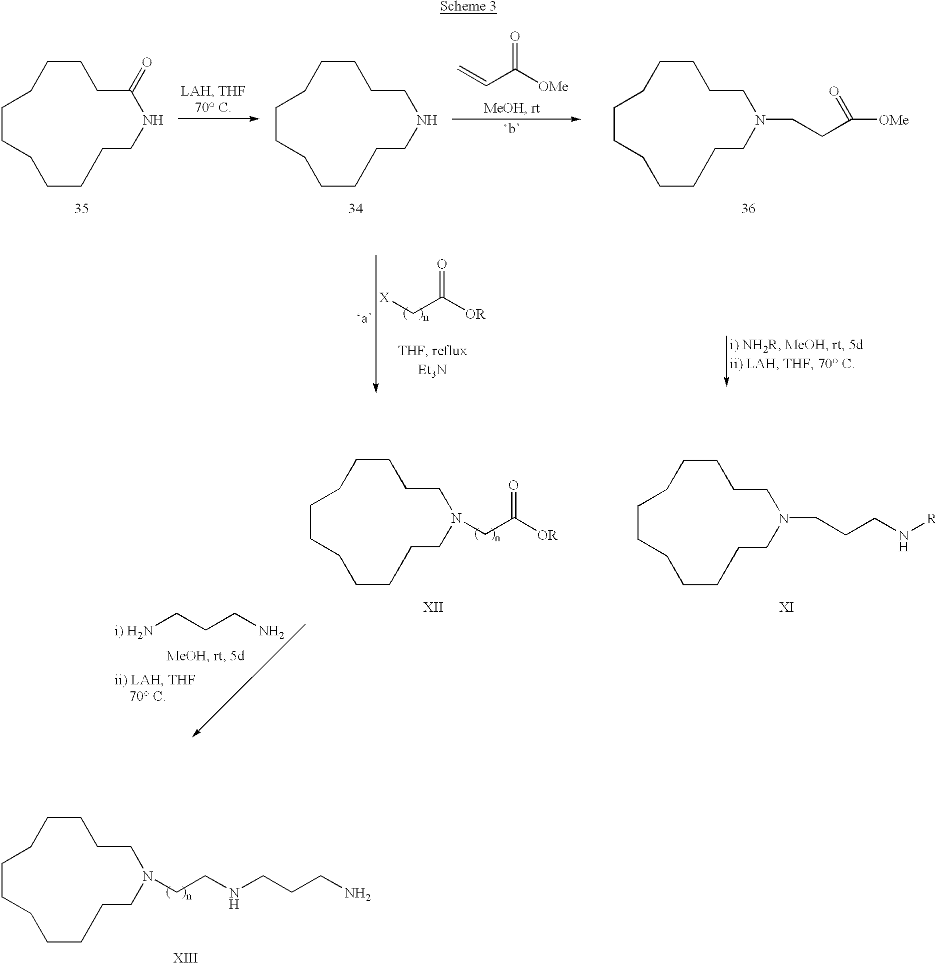

Compounds of this invention or for use in this invention are generally water soluble and may be formed as salts. In such cases, pharmaceutical compositions in accordance with this invention may comprise a salt of such a compound, preferably a physiologically acceptable salt such as the HCl salt. Other suitable salts are known in the art. Pharmaceutical preparations will typically comprise one or more carriers acceptable for the mode of administration of the preparation, be it by injection, inhalation, topical administration, lavage, or other modes suitable for the selected treatment. Suitable carriers are those known in the art for use in such modes of administration.

Pharmaceutical compositions in accordance with this invention or for use in this invention may be administered to a patient by standard procedures, including topical, oral, inhalation, intramuscular, intravenous, or intraperitoneal administration. For contraceptive indications, administration may be by direct application to the uterus, for example by a uterine wash, implant, or intrauterine device. Dosage and duration of treatment will be determined by the practitioner in accordance with standard protocols and information concerning the activity and toxicity of the chosen compound.

Compounds or pharmaceutical compositions in accordance with this invention or for use in this invention may be administered by means of a medical device or appliance such as an implant, graft, prosthesis, stent, etc. For example, a stent may be coated with such a composition for inhibition of restenosis or atherosclerosis. Also, implants may be devised which are intended to contain and release such compounds or compositions. An example would be an implant made of a polymeric material adapted to release the compound over a period of time.

### EXAMPLES

### A. SCREENING FOR INHIBITORS OF CELLULAR INVASION

Matrigel™ is an extract of the transplantable Engelbreth-Holm-Swarm murine sarcoma that is rich in laminin, collagen IV, entactin and heparin sulphate proteoglygen. At 4° C. Matrigel is liquid, but at 37° C. it polymerizes in a cation-dependent manner to form a semisolid gel. These physical properties have been exploited to develop two widely used cellular invasion procedures. In the “outgrowth” procedure, cells are suspended in liquid Matrigel followed by gelling. Invasion is then monitored morphologically as the cells form outgrowths into the gel (Bae, S. N., et al. (1993) Breast Cancer Res. Treat, 24:241-255). In the “Boyden chamber” procedure, Matrigel is pre-gelled upon a porous filter support. Cells are then placed on the Matrigel and invasion is quantified by determining the number of cells that cross to the other side of the basement membrane/filter barrier, usually in response to a chemotactic agent (Price, J. T. and Thompson, E. W. (1999) Meth. Mol. Biol. 129:231-250). The deficiency in the “outgrowth” procedure is the need to visually monitor changes in cell morphology. The “Boyden chamber” procedure generates quantitative data, but like the “outgrowth” procedure it is unable to discriminate between agents that affect invasion and cell viability or cell adhesion.

The inventors herein have recognized that a significant proportion of crude biological extracts cause cell death because of the high concentrations of salt and other toxic molecules present in many extracts. However, the assay of this invention is quantitative and eliminates those agents that prevent cell adhesion or which are cytotoxic. The assay of this invention scores a “positive” hit for cells that remain attached to the matrix, show a decrease in invasion and remain viable from the beginning to end of the procedure.

The MDA 231 human breast carcinoma cell line is highly invasive and metastatic, These cells attach to Matrigel within 15 min and begin to invade and migrate into the reconstituted basement membrane matrix within 2-4 hours in a manner that can be assessed morphologically. In contrast, MDA 453 human breast cancer cells are much less metastatic and while they rapidly attach to Matrigel, they do not invade the matrix within 4 hours. Differential invasive properties between the two cells lines such as these may be used to develop optimal conditions for recovery of non-invasive cells in the method of this invention. In this example, recovery of the less invasive MDA 453 cell line was greater than 5 fold the recovery of the more invasive MDA 231 line.

Suitability of the assay of this invention was tested in drug screening using a selection of crude extracts from marine sponges. 230 extracts were tested at 50-100 μg/ml. 228 extracts showed readings close to or below those of negative controls containing dimethylsulfoxide (DMSO) (0.025). However, two extracts showed strong activity, higher than a positive control using LY294002, a phosphatidylinositol 3-linase inhibitor known to inhibit invasion. The active compounds were purified from the first extract, as described below using the assay to guide fractionation. The second active extract resembled the first one morphologically and its extract was determined to contain the same active compounds. The active compounds were identified as motuporamines, a family of macrocyclic alkaloids with a spermidine-like substructure.

Cell Culture

Human breast carcinoma MDA 231 and MDA 453 cells obtained from the American type culture collection (Bethesda Md.) and were cultured in a 1:1 (v:v) mixture of Dulbecco's modified Eagle medium and F12 culture medium (DMEM/F12) supplemented with 5% fetal bovine serum (FBS), insulin (5 μg/ml) and gentamycin (50 U/ml). Human umbilical vein endothelial cells (HUVEC) were isolated by flushing fresh umbilical cord veins with 50 ml Roswell Park Memorial Institute (RPMI) medium followed by 20 ml collagenase A in RPMI (0.13 mg/ml). The cords were then filled with collagenase A and incubated at rt for 30 min. After massaging the cords to dislodge cells, the contents were flushed with 30 ml RPMI, harvested and centrifuged at 1200 rpm for 10 min. The cell pellet was washed with RPMI and suspended in medium complete with trace elements (MCDB) supplemented with 10% fetal calf serum, 10% iron-supplemented fetal calf serum, 16 U/ml heparin, 20 μg/ml endothelial cell growth supplement (ECGS), 2 mM glutamine and 100 U/ml each penicillin and streptomycin. For the initial passage, cells were plated onto dishes coated with 0.2% gelatin to promote HUVEC attachment. Subsequent passages were performed using standard tissue culture treated dishes. All cells were maintained at 37° C. in 5% CO₂.

Marine Organism Collection and Extract Preparation

Approximately 250 g each of marine sponges were collected by hand from tropical Pacific Ocean reefs at a depth of about 15 m off Motupore and Madang in Papua New Guinea. Samples were deep frozen on site and transported over dry ice. Voucher samples of each were kept in methanol at −20° C. at The University of British Columbia, Canada, for taxonomic identification. Samples of Xestospongia. exigua were deposited at the Zoological Museum of Amsterdam (ZMA POR 11521). Extracts were prepared by homogenizing 200 g of each sponge sample in methanol. The homogenates were filtered and concentrated under vacuum to give a gummy residue. About 1 mg was dissolved in 100 μl DMSO and used in the invasion inhibitor assay.

Assay for Invasion Inhibitors

Matrigel (Collaborative Biomedical Products, Bedford, Mass.) was diluted 1:1 in ice-cold DMEM/F12 and 50 μl was pipetted into wells of ice-cold 96-well cell culture plates (Falcon) using ice-cold pipette tips, avoiding the sides of each well. The matrix was then allowed to gel and then dry overnight in a dry 37° C. incubator followed by rehydration with 100 μl of growth medium 1 h prior to cell plating. This treatment produces a matrix that is less prone to breakage. On top of the Matrigel, was added 100 μl medium warmed to 37° C., with or without 1 μl of sponge extract dissolved in DMSO, followed by 100 μl medium containing 60.000 MDA 231 cells. Addition of 1 μl of DMSO served as a negative control. 50 μM μM LY294002, a phosphatidylinositol 3-kinase inhibitor known to inhibit invasion, served as a positive control. The cells were then incubated for 2.5 h to allow invasion to take place.

After incubation, cells had either invaded the Matrigel or failed to invade and settled on the surface of the Matrigel. Most of the cell culture medium was removed without disturbing the cells. This may be accomplished using a hand-held pipettor, but more consistent results are achieved using the aspiration function of a Bio-Tek Elx405 96-well plate washer with the aspiration needle positioned about 2 mm above the surface of the Matrigel. The cells that failed to invade were recovered by detaching them from the surface of the Matrigel by incubation with 200 μl of 0.125% trypsin in Hank's Balanced Sit Solution for 30 min at 37° C. This enzymatic treatment detaches cells that are attached to the upper surface of the Matrigel but it does not degrade the matrix and does not release cells that have invaded the gel. The cells were then suspended by pipetting up and down three times using the 100 μl setting of a hand-held pipettor, and 100 μl was withdrawn and transferred to fresh plates without Matrigel containing 100 μl medium supplemented with 30% fetal calf serum to inactivate the trypsin. The cells were then incubated overnight to allow attachment of cells to the plastic surface. Live cells were measured using the MTT assay: the cell culture medium was replaced with 100 μl fresh medium and 25 μl of a 5 mg/ml solution of 3(4,5-dimethylthiazol-2yl)-2,5-diphenyltetrazolium bromide in phosphate-buffered saline. After 2 hr at 37° C., the solution was removed and 100 μl of 20% sodium dodecyl sulfate dissolved in dimethylformamide: water (1:1) was added to lyse the cells. After overnight incubation at 37° C., absorbance was measured at 570 min.

### B. ISOLATION AND SYNTHESIS OF MOTUPORAMINES

General Chemical Methods

Low and high resolution FABMS were recorded on a Kratos Concept II HQ mass spectrometer with xenon as the bombarding gas and a thioglycerol sample matrix.

Merck Type 5554 silica gel plates and Whatman MKC18F plates were used for analytical thin layer chromatography. Reversed-phase HPLC purifications were performed on a Waters 600E System Controller attached to a Waters 410 Differential Refractometer. All solvents used for HPLC were Fisher HPLC grade.

Isolation of Motuporamines

A portion of a frozen specimen of Xestospongia exigua (86 g) was cut into small pieces, immersed in and subsequently extracted repeatedly with methanol (3×150 ml). The combined methanolic extracts were concentrated under vacuum and then partitioned between ethyl acetate (3×70 ml) and H₂O (200 ml). The aqueous layer, which exhibited activity in the invasion assay, was next extracted with n-butanol (3×70 ml). The combined butanol extracts were concentrated under vacuum to yield 1.16 g of brown oil. The active n-butanol soluble material was separated by repeated chromatography on Sephadex LH-20 eluting first with methanol (yielding 118.4 mg of active material) as the eluent and then 20:5:2 ethyl acetate/methanol/H₂O to give 55.6 mg of a pale amorphous solid that stained with ninhydrin and was active in the invasion assay. The amorphous solid consisted of a single class of compounds with a spermidine-like substructure, comprising the known Motuporamines A-C and several new motuporamines. Motuporamine C was the major component (>90%) in the sample.

The compounds were separated at high dilution (<0.1 mg per injection) on reversed-phase HPLC using a Whatman Magnum-9 Partisil 10 ODS-3 column, with 2% trifluoroacetic acid (TFA) in 11:9 methanol/H₂O as the eluent. This HPLC fractionation gave a pure sample of Motuporamine A, a fraction containing a mixture of B and D (consisting of at least 4 methyl branched analogs), a pure sample of Motuporamine C, a fraction containing >90% Motuporamine E, plus two other olefins isomeric with E, and a pure sample of acetylmotuporamine C and trifluoracetylmotuporamine C which may both be artifacts of isolation. Except for Motuporamine C, all other compounds were isolated in sub-mg quantities. All of the motuporamines isolated were the ammonium TFA/water salts.

In a subsequent large scale isolation, the procedure was improved by redissolving the active n-butanol extract in H₂O, adding 5N NaOH to pH>12 and then extracting with CH₂Cl₂. The active CH₂Cl₂ extract was then chromatographed as above. A total of 379.7 mg of motuporamines was isolated from 625 g of frozen sponge.

Motuporamine A was isolated as a pale oil; positive ion HRFABMS [M+H]⁺ m/z 298.3229 (C₁₈H₄₀N₃, calcd 298.3222). Motuporamine B, Motuporamine D, and methyl positional isomers of Motuporamine D were isolated as a white amorphous solid; positive ion HRFABMS [M+H]⁺ m/z 312.3387 (C₁₉H₄₂N₃, calcd 312.3379). Motuporamine C was isolated as a pale amorphous solid; positive ion HRFABMS [M+H]⁺ m/z 324.3380 (C₂₀H₄₂N₃, calcd 324.3379). Motuporamine E and two additional olefins isomeric with E were isolated as a pale oil; positive ion HRFABMS [M+H]⁺ m/z 310.3212 (C₁₉H₄₀N₃ calcd 310.3222). Acetylmotuporamine C was isolated as a white amorphous solid; positive ion HRFABMS [M+H]⁺ m/z 366.3484 (C₂₂H₄₄N₃ calcd 366.3484). Trifluoracetylmotuporamine C was isolated as a white amorphous solid [M+]⁺ m/z 420.3191 (C₂₂H₄₁N₃OF₃ calcd 420.3202).

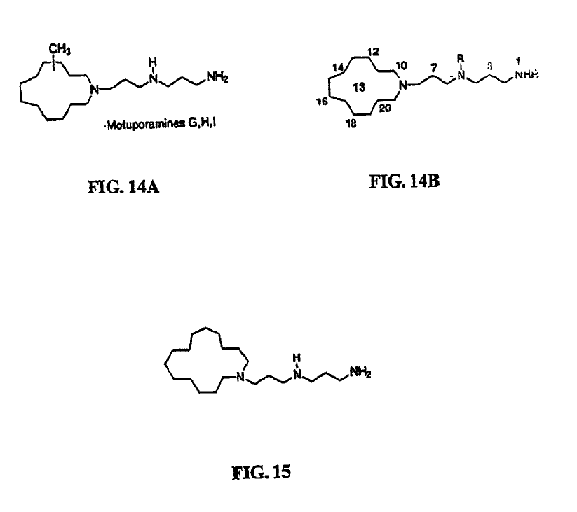

Motuporamines G, H, and I were all obtained in very small quantities. Each of them gave [M+H]⁺ ions in the HRFABMS that corresponded to molecular formulas of C₁₉H₄₁N₁₃. Analysis of the NMR data obtained for each compound showed that they were all members of the motuporamine family with the same diamine side chain found in all the other natural analogs and that they also had a saturated macrocyclic amine fragment. The feature that set Motuporamines G, H, and I apart was the presence of a doublet methyl resonance at δ˜0.98 in the ¹H NMR spectrum of each, which is assigned to a methyl branch in the macrocyclic amine of each compound, making them methylated analogs of Motuporamine A. It was possible to rule out methyl branches at C-10 and C-11. Since there are three distinct isomers, they must each have general structure as shown in FIG. 14, where the branches are individually located between C-12 and C-15.

Chemical Modifications of Motuporamines

Acetylation: A sample of the mixture of motuporamines (89.2 mg) was dissolved in 2 ml of 3:1 pyridine/acetic anhydride and stirred at rt for 16 h. Removal of the solvent in vacuo gave a mixture of diacetylated products that were partially separated by semi-preparative reversed-phase HPLC, using a Whatman Magnum-9 Partisil 10 ODS-3 column, with 2:3 MeCN/0.6% TFA/H₂O as the eluent. The diacetylated Motuporamines A (eluting first) and C (1.5 and 95.8 mg, respectively) were obtained as pale clear oils. Three additional fractions were also obtained. The first of these contained diacetylmotuporamine E. This fraction was further purified on reversed-phase HPLC, using a Whatman Magnum-9 Partisil 10 ODS-3 column, with 0.39% TFA in 35:65 MeCN/H₂O as eluent, to yield 1.2 mg of the pure compound as a pale oil. The next eluting fraction, contained a mixture of diacetylmotuporamine B and the diacetyl methyl branched analogs of Motuporamine A, and was further fractionated on HPLC using the same conditions as for diacetylmotuporamine E above. Pure diacetylmotuporamine B (0.4 mg), and two fractions (0.8 and 0.6 mg) each containing two methyl branched adducts of Motuporamine A were obtained. The slowest eluting fraction, eluting after diacetylmotuporamine C, was purified using a Whatman Magnum-9 Partisil 10 ODS-3 column, with 0.39% TFA in 33:67 MeCN/H₂O as eluent, to yield 0.5 mg of the formamide of diacetylmotuporamine C as a pale oil. Additional diacetylmotporamines were collected in each of the HPLC fractionations described. All diacetylmotporamines were isolated as the ammomium TFA/water salts.

Diacetylmotuporamine A and C were previously known. However, diacetylmotuporamine B was previously described incorrectly (Williams, D. E., et al. [supra]). It is now shown that diacetylmotuporamine B is isolated as a pale clear oil; positive ion HRFABMS [M+H]⁺ m/z 396,3580 (C₂₃H₄₆N₃O₂. calcd 396.3590). Methyl branched forms were isolated as two pale oils that each contained Motuporamine D and isomeric methyl branched analogs of diacetylmotuporamine A; positive ion HRFABMS [M+H]⁺ m/z 396.3599 (C₂₃H₄₆N₃O₂, calcd 396.3590). Diacetylmotuporamine C formamide was isolated as a pale clear oil; positive ion HRFABMS [M+H]⁺ m/z 436.3538 (C₂₅H₄₆N₃O₃, calcd 436.3539). Diacetylmotuporamine E was isolated as a pale clear oil [M+H]⁺ m/z 394.3427 (C₂₃H₄₄N₃O₂, calcd 394.3434).

Hydrogenation: hydrogenation of the motuporamine mixture with H₂/Pd/C gave a mixture of fully reduced motuporamines that exhibited strong activity in the invasion assay. An example is Dihydroynotuporamine C as shown in FIG. 15 Dihydroynotuporamine C was isolated as an amorphous solid; ¹H NMR (400 MHz, MeOH-d₄) δ3.24 (m, 2H), 3.10-3.21 (m, 8H), 3.05 (m, 2H), 2.17 (m, 2H0, 2.08 (m, 2H), 1.72 (m, 4H), 1.46 (m, 8H), 1.36-1.42 (m, 12H) ppm; ¹³C NMR (100 MHz, MeOH-d₄) δ53.3, 52.9, 46.0, 46.0, 37.8, 27.7, 27.4, 27,4, 27.3, 25.5, 25.4, 22.9, 22.6 ppm; positive-ion HRFABMS [M+H]⁺ m/z 326.3532 (C₂₀H₄₄N₃, calcd 326.3535).

HCl Hydrolysis: HCl hydrolysis of diacetylmotuporamine C (which is inactive in the invasion assay) gave Motuporamine C that exhibited strong activity in the invasion assay (FIG. 6).

Preparation of Motuporamines, Motuporamine Analogs with Side Chain Modifications and Other Motuporamine Analogs from Dialkyl, Cyclo- and Macrocycloamines

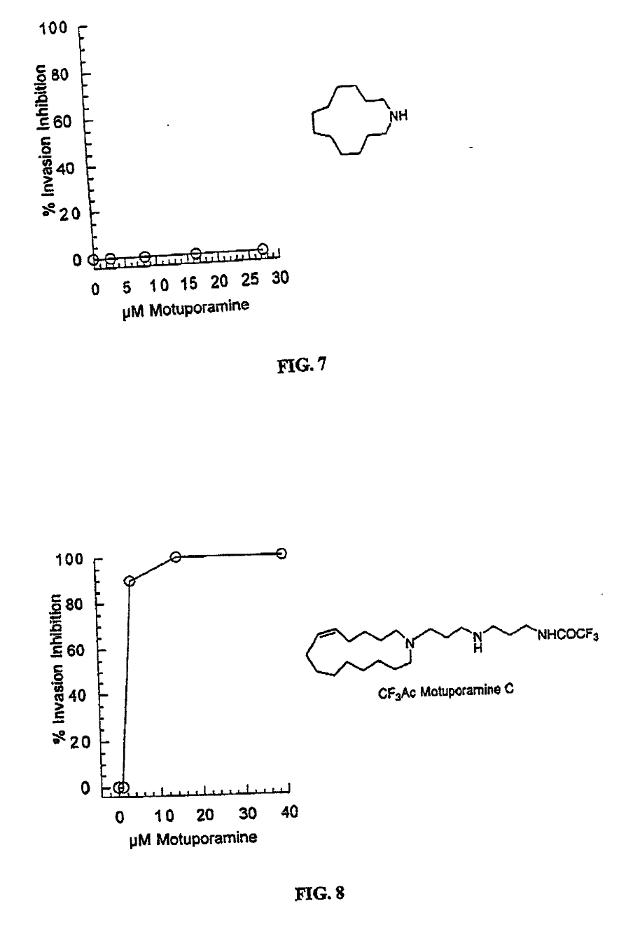

A secondary amine (e.g. about 1.5 mmole) (e.g. the macrocyclic amine shown in FIG. 7) may be reacted with methyl acrylate (e.g. about 1.6 mmole) in MeOH (e.g. 10 ml at rt for 16 h). After removal of the solvent and excess reagent in vacuo, the resulting amino ester may be reacted with (e.g a 10 fold excess) of a diamino alkane (e.g. ethylenediamine, 1,3-diaminopropane, 1,4-diaminobutane, spermidine) in MeOH (e.g. 10 ml at rt for 4-5 days). Evaporation of the solvent and excess reagent in vacuo provides an amide. The amide may be reduced with lithium aluminum hydride (LAH) (e.g. 1.3 mmole in THF (7 ml) at 70° C. for 16 h). The excess LAH may be quenched by dropwise addition of H₂O and the reaction mixture partitioned between H₂O (e.g. 10 mL) and Et₂O (e.g. 3×4 ml) with the pH kept at >12 (e.g. by addition of 1N NaOH when necessary). HPLC may be used for further purification. For example, an amorphous TFA/H₂O salt of an analog may be prepared by semi-preparative reversed-phase HPLC, using a Whatman Magnum-9 Partisil 10 ODS-3 column, with 2% TFA in 11.9-9:11 MeOH/H₂O as eluent in a procedure modified from Goldring, W. P. D. and Weiler. L. [supra].

Preparation of Motuporamines and Motuporamine Analogs from 2-Azacyclo-alkyl-ones

A 2-azacyclo-alkyl-one (1.52 mole) (2-azacyclotridecanone, 2-azacyclononanone or 4-azatricyclo[4.3.1.13,8]undecano-5-one) was reduced with LAH (1.3 mmole) according to the procedure outlined above for the reduction of an amide. The resulting macrocyclic amine (yield generally 98%) was reacted with methyl acrylate and subjected to the remaining procedure as described above to provide Motuporamine A.

Preparation of Motuporamine Analogs with Side Chain Modifications

A secondary amine (e.g. the macrocyclic amine in FIG. 7; 1.52 mmole) was coupled with a methylhaloalkyl ester (1.57 mmole) (methylchloroacetate or methyl-4-iodobutyrate) in THF (12 ml) under reflux in the presence of Et₃N (4.56 mmole) for 3 h following a procedure described in Sbiozaki, M., et al. (1984) Tetahedron 40:1795-1802. After Et₂O extraction between H₂O (30 mL) and Et₂O (3×8 ml) the resulting ether soluble α or γ- amino ester was reacted with a 10-fold excess of diaminoalkane as described above to provide a Motuporamine A analog.

Preparation of Carbazole Analog

Carbazole (1.52 mmole) was dissolved in THF (10 ml) and NaH (1.56 mmole) added. The mixture was left stirring for 2 hours at room temperature (rt) and after the addition of methyl acrylate, the reaction mixture was left stirring at rt for an additional 16 h. Excess NaH was quenched dropwise with H₂O and the reaction mixture extracted between H₂O (22 ml) and Et₂O (3×7 ml). The resulting β-amino ester was then reacted with a 10 fold excess of 1,3-diaminopropane and the resulting amide was reduced with LAH, as described above to provide the compound shown in FIG. 12.

The compound shown in FIG. 12 was isolated as a yellow amorphous solid, ¹H NMR (400 MHz, MeOH-d₄) δ8.07 (d, J=7.5 Hz, 2H), 7.52 (d, J=7.9 Hz, 2H), 7.44 (t, J=7.9 Hz, 2H), 7.20 (t, J=7.5 Hz, 2H), 4.50 (t, J=6.7 Hz, 2H), 2.94-3.04 (m, 6H), 2.27 (m, 2H0, 1.97 (m, 2H) ppm; ¹³C NMR (100 MHz, MeOH-d₄) δ141.5, 127.0, 124.3, 121.3, 120.3, 109.7, 46.9, 45.8, 40.6, 37.7, 26.9, 25.3 ppm; positive-ion HRFABMS [M+H]³⁰ m/z 282.1972 (C₁₈H₂₄N₃, calcd 282.1970),

### C. ACTIVITY OF MOTUPORAMINES

Inhibition of Cellular Invasion

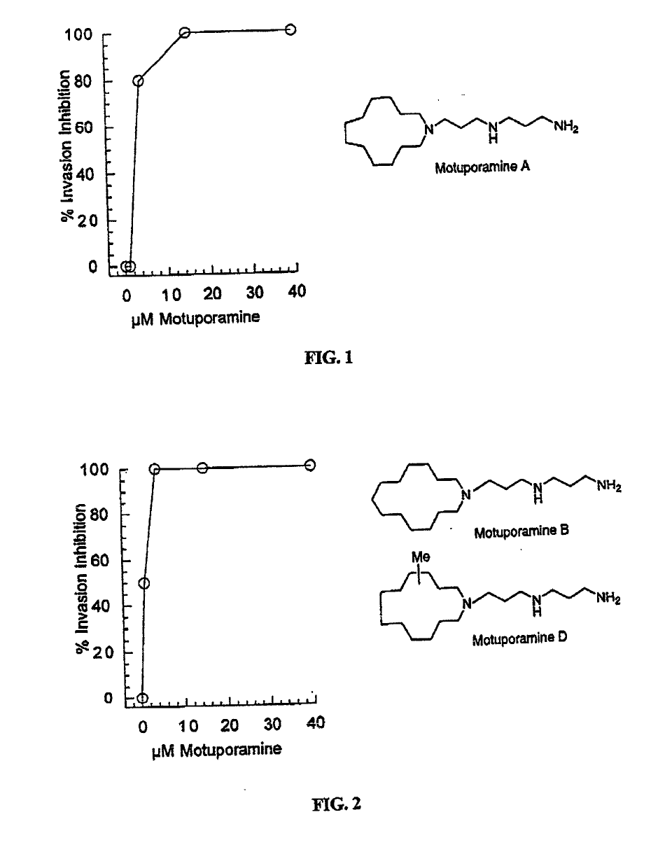

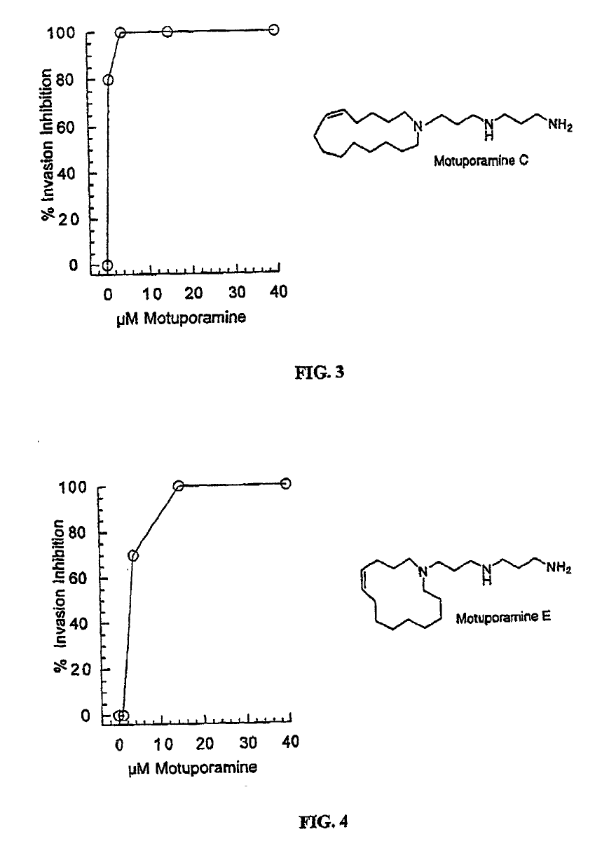

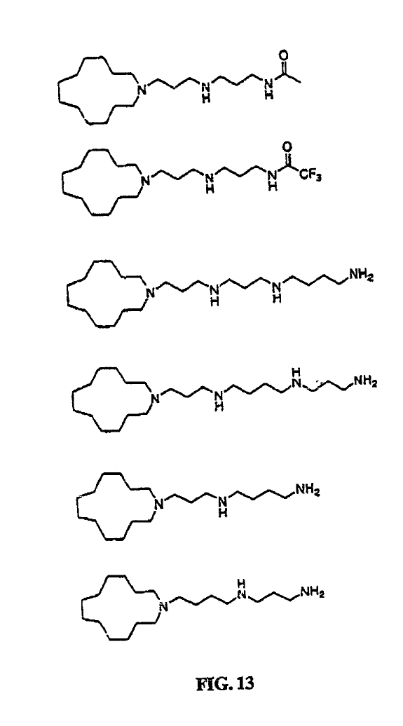

Different concentrations of Motuporamine A, Motuporamine B. Motuporamine C, a mixture of the B and D forms and a mixture of Motuporamine G, H, and I, were tested in the invasion inhibition assay. All inhibited invasion. Motuporamine C (IC₅₀=1 μm; FIG. 3), Motuporamine A (IC₅₀=3 μM; FIG. 1), the B and D mixture (IC₅₀=3 μM; FIG. 2), Motuporamine B and the G, H, I mixture showed similar activity. FIGS. 4, 5, 6, and 8-12 also show inhibition by various compounds of his invention. The IC₅₀ values for the compounds shown in FIG. 13 were from about 1-10 μm.

Low Cytotoxicity

The cytotoxic effects of Motuporamine C were examined by incubating cells in the presence of different concentrations of the compound for 24 h. Motuporamine C was washed away and cell proliferation was measured for up to 5 additional days. Motuporamine C at a concentration close to the IC₅₀ for invasion inhibition (1.6 μM), had no detectable effects on cell proliferation. Higher concentrations of 4 and 8 μM showed mild inhibition of cell proliferation. Therefore, Motuporamine C shows little or no toxicity towards proliferating MDA 231 cells at the low micromolar concentrations at which it inhibits invasion. Likewise, the compounds shown in FIGS. 1, 2, 4, 5, 6, and 8-13 have low cytotoxicity.

Activity of Motuporamine Analogs

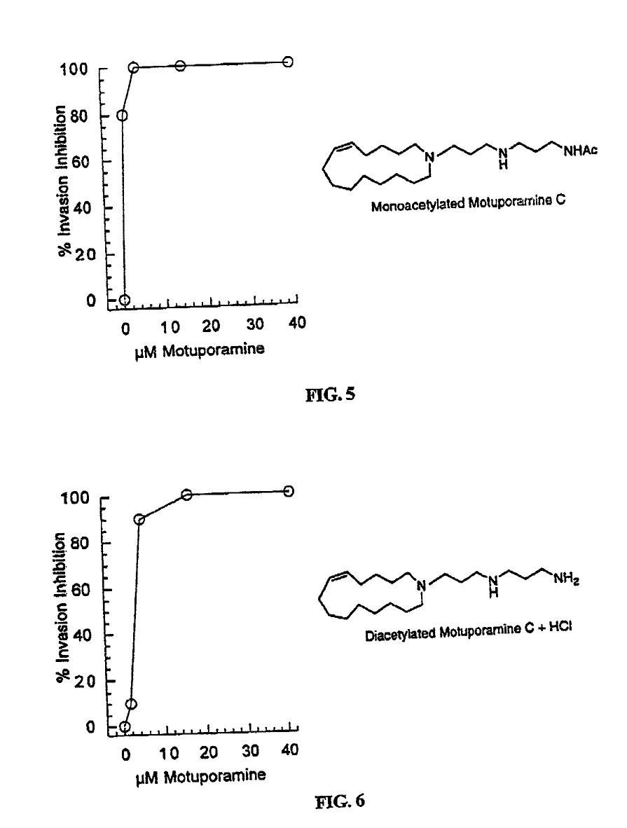

A conspicuous feature of motuporamines is their spermidine-like tail, which is positively charged at physiological pH. Synthetic and semisynthetic motuporamine analogs were prepared and tested at different concentrations in the invasion inhibition assay. A motuporamine A analog lacking the spermidine-like tail was completely inactive (FIG. 7). The terminal amino group of Motuporamine C was acetylated (monoacetylated Motuporamine C; FIG. 5) or substituted with trifluoroacetate (CF₃Ac Motuporamine C; FIG. 8). Both compounds were highly active, showing that a charged amino group is not required at this position.

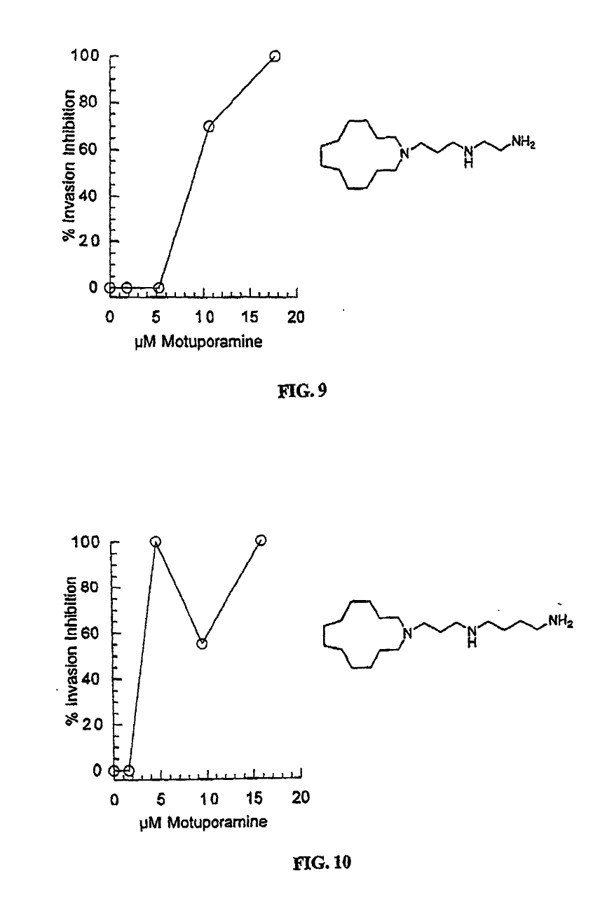

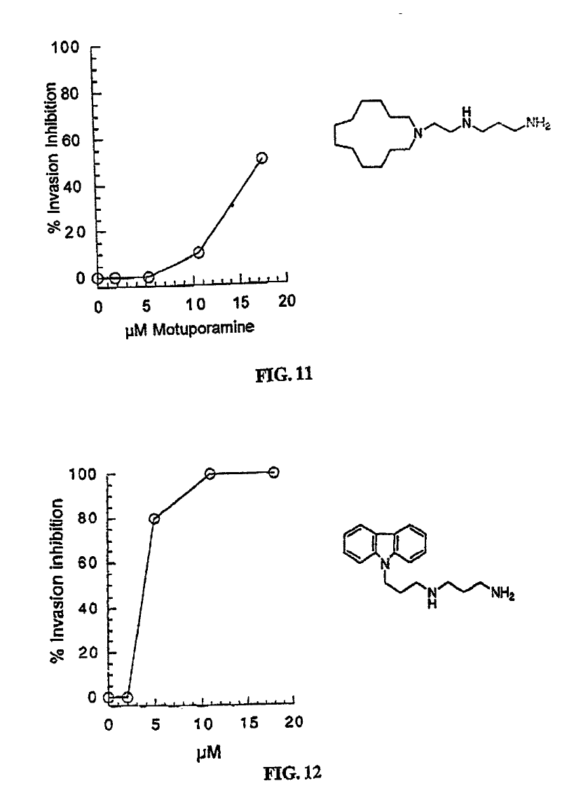

The number of carbons between the nitrogens of the tail was varied. A compound with only two carbons adjacent the terminal amino group showed strong activity (FIG. 9) and a compound with four carbons instead of three also showed strong activity (FIG. 10). A compound with only two carbons instead of three adjacent the ring (FIG. 11) is active.

A second feature of the naturally occurring motuporamines is the simple 13-15 membered ring. Analysis showed that Motuporamine C with a 15-membered ring is slightly more active than the 14-membered Motuporamine E and the 13-membered Motuporamine A. A series of synthetic analogs with different ring sizes were prepared and tested for invasion inhibition. A compound containing a 5-membered ting and a compound with a 6-membered ring coupled to the spermidine-like tail showed no activity. A compound having a fully unsaturated, multiple ring structure demonstrated potent activity (carbazole analog; IC₅₀=3 μM; FIG. 12).

Motuporamines Inhibit Cancer Cell Spreading

MDA 231 cells are highly invasive. They rapidly attach to the Matrigel and within 4 hours have taken on a fusiform morphology and have migrated into the gel. Incubation of cells with Motuporamine C prevents the formation of invasive, fusiform cells on Matrigel. The motuporamine concentration that completely inhibited invasion (5 μM) did not cause the cells to detach from the matrix. Rather, the cells remained attached but were rounded, sitting on top of the matrix.

The observation that Motuporamine C caused MDA 231 cells to remain rounded on Matrigel suggested inhibition of cell spreading. The cells were plated on rigid tissue culture plastic in the presence of serum, which contains spreading factors (Ham). Under these conditions, most MDA 231 cells spread on the plastic within 4 h in the absence of Motuporamine C. The spreading cells displayed large, flattened lamellae with prominent, continuous ruffling edges. These ruffles, which appear black in phase contrast microscopy represent sites of dynamic actin-mediated membrane movement in both spreading and migrating cells. Increasing doses of Motuporamine C decreased cell spreading with most cells appearing rounded at 5 μM. These rounded cells remained attached to the tissue culture plastic by small lamellae that had small, discontinuous ruffles.

MDA 231 cells were also pre-spread for 20 hours on tissue culture plastic and then treated with Motuporamine C (5 μM) for 4 hours. Phase contrast microscopy revealed that the cells became slightly refractile, indicative of slight retraction, but did not round-up. As determined by staining with rhodamine-phalloidin, treatment with Motuporamine C causes subtle changes to the actin cytoskeleton: actin stress fibers are still visible but there are often small discrete “buttons” of actin localization at cell edges in place of continuous raffles. In contrast, treatment with the f-actin disrupting agent cytochalasin D (CD; 1 μg/ml) causes the cells to round up completely and causes complete disassembly of the actin filaments. Treatment of cells with Motuporamine C for up to 48 hours did not cause significant cell rounding of the cells already attached and spread on the surface.

Motuporamines Inhibit Cancer Cell Migration

The effects of Motuporamine C on cell spreading and actin ruffling indicates invasion inhibition by decreasing cell migration. A sterile toothpick was drawn across a confluent monolayer of MDA 231 cells, leaving a cell-free gap of about 100 μm. Closure of the gap by cell migration was monitored by microscopy. In the absence of Motuporamine C, cells migrated into the gap and essentially closed it within 24 hours. These cells had broad lamellar ruffles along their leading edges throughout which were condensations of actin. In contrast, there was still a considerable gap present at 24 hours when cells were exposed to Motuporamine C (5 μM), indicating that Motuporamine C slows cell migration. These cells had only small and discontinuous ruffles along their leading edges and contained only small discrete patches of actin, looking similar to the “button-like” condensations observed in pre-spread cells.

Endothelial Sprouting Assay

Endothelial sprouting was assessed by a modification of Nehls, V. and Drenckham, D. (1995) Microvasc. Res. 50:311-322). Microcarrier beads coated with denatured collagen (Cytodex 3, Sigma) were seeded with HUVEC. When the cells reached confluence on the beads, equal numbers of HUVEC-coated beads were embedded in fibrin gels in 96-well plates. For preparation of fibrin gels, bovine fibrinogen was dissolved in MCDB medium at a concentration of 2.5 mg/ml. Aprotinin was added at a concentration of 0.05 mg/ml and the solution was filtered through a 0.22 μm filter. The fibrinogen solution was supplemented with 15 ng/ml vascular endothelial growth factor (VEGF) with or without Motuporamine C. As a control, fibrinogen solution without VEGF or Motuporamine C was used. Following transfer of the fibrinogen solution to 96-well plates, HUVEC-coated beads were added at a density of 50 beads per well and clotting was induced by the addition of thrombin (1.2 U/ml). After clotting was complete, gels were equilibrated with MCDB medium containing 5% fetal calf serum at 37° C. After 60 min of incubation, the medium was replaced with the same medium with or without Motuporamine C. After 3 days of incubation with daily changes of the medium, the number of capillary-like tubes formed per microcarrier bead (sprouts/bead) was counted by microscopy. Only sprouts greater than 150 μm in length and composed of at least 3 endothelial cells were counted.

Chick Chorioallantoic Membrane Assay (CAM) for Angiogenesis

Fertilized White Leghorn chicken eggs were incubated at 37° C. under conditions of constant humidity. On embryonic day 6, the developing chorioallantoic membrane (CAM) was separated from the shell by opening a small circular window at the broad end of the egg above the air sac. After removal of the inner membrane, the opening was sealed with Parafilm and the eggs were incubated for 2 more days. Motuporamine C was prepared in PBS supplemented with 30 ng/ml VEGF. On day 8, 20 μl was loaded onto 2 mm³ gelatin sponges (Gelfoam, Pharmacia Upjohn) that were placed on the surface of the developing CAM. Sponges containing vehicle alone (20 μl PBS) were used as negative controls whereas sponges containing 20 μl of 30 ng/ml VEGF in PBS were used as positive controls. Eggs were resealed and returned to the incubator. On day 10, images of CAM were captured digitally using an Olympus SZX9 stereomicroscope equipped with a Sport RT digital imaging system (Diagnostics Instruments).

Motuporamines Inhibit Angiogenesis In Vitro and In Vivo

Motuporamine C inhibits angiogenesis as shown in the in vitro endothelial sprouting assay and in the in vivo chick chorioallantoic membrane (CAM) assay. In the endothelial sprouting assay, human umbilical vein endothelial cells (HUVEC) are seeded onto collagen-coated beads. Exposure to vascular endothelial growth factor stimulates the formation of capillary-like tubes called sprouts, whose number and length may be measured over time by microscopy. In this assay 2.5 μM Motuporamine C showed clear inhibition of sprout formation in response to vascular endothelial growth factor (VEGF, 15 ng/ml). AT 5 μM, Motuporamine C showed complete inhibition of sprouting.

Motuporamine C had no effect on survival of confluent HUVEC at concentrations up to 10 μM and for up to 72 hours. Also, Motuporamine C does not decrease the proliferation rate of HUVEC and may enhance proliferation of HUVEC slightly. Therefore, Motuporamine C does not inhibit angiogenesis through toxic or antiproliferative effects. Rather, Motuporamine C inhibited HUVEC migration as assessed using a modified Boyden chamber assay. The compound inhibited the migration of HUVEC towards the VEGF (15 ng/ml) at both 2.5 μM and 5 μM.

Motuporamine Inhibit Tumour Growth in Vivo

Motuporamine C administered (i.p.) daily to mice bearing Lewis Lung and SCCVII tumours at a dose of 10-15 mg/kg/day caused a significant decrease in tumour growth compared to vehicle dosed control animals. Drug administration started when tumours were 50-200 mm3 in volume and resulted in a 25-50% reduction in tumour growth rate compared to controls.

Motuporamine Inhibit the Development of Tumour Metastases in Vivo

Motuporamine C administered (i.p.) at 15 mg/kg/day to C57 mice implanted with a primary Lewis lung tumour showed a decreased incidence of lung metastases compared to vehicle treated control animals. Primary tumours were grown to a specific size and then irradiated permitting the animals to survive long enough for lung metastases to be quantified.

Motuporamine Inhibit Neuronal Migration

Synthetic dihydromotuporamine (DiH-MPC, 5 μM) affected neuronal migration in a well-characterized insect embryo model. DiH-MPC slowed stereotypical migration of axonal growth cones whose ultimate synaptic targets are in the central nervous system (CNS). Importantly, there were no steering or pathfinding deficits; only the rate of nerve growth cone migration at the ends of the developing axons was inhibited. Therefore, the motuporamines have application in situations where reduced axonal sprouting would be of benefit after CNS wounding. This is of particular interest in spinal cord lesions where wound-induced axonal sprouting and migration (i.e. plasticity) leads to the inappropriate intermingling of neuronal pathways, which is a major impediment to clinically efficacious CNS regeneration.

Although the foregoing invention has been described in some detail by way of illustration and example for purposes of clarity of understanding, it will be readily apparent to those of skill in the ant in light of the teachings of this invention that changes and modification may be made thereto without departing from the spirit or scope of the appended claims. All patents, patent applications and publications referred to herein are hereby incorporated by reference.

What is claimed is:

1. A pharmaceutical composition for inhibiting cellular invasion or angiogenesis, wherein the composition comprises one or more cellular invasion or angiogenesis inhibiting compounds of formula I or II or pharmaceutically acceptable salts thereof, and a pharmaceutically acceptable carrier, wherein formula I is: 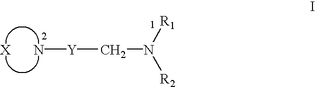
wherein:
X is a saturated, or unsaturated linear, branched, or partially cyclized alkyl chain of between eleven and thirty carbons optionally substituted with one or more substituents selected from the group consisting of: epoxide, ketone, thiocarbonyl, oxime, —OH, —OR, —O₂CR, —SH, —SR, —SOCR, —NH₂, —NHR, —NR₂, —NR³+, —NHCOR, —I, —Br, —Cl, —F, —CN, —CO₂H, —CO₂R, —CHO, —COR, —CONH₂, —CONHR, NRCOR, —CONR₂, —COSR, —NO₂, —OSO₃H, —SO₃H, —SOR and —SO₂R; wherein one or more CH₂ groups if present in the alkyl chain, is optionally replaced by a moiety selected from the group consisting of: O, S, NH and NR; and wherein one or more C and CH groups if present in the alkyl chain, is optionally replaced with NH or NR;
R₁ and R₂ are independently selected from the group consisting of: hydrogen; methyl; a linear, branched, or cyclic saturated, or unsaturated alkyl group containing one to ten carbons optionally substituted with one or more substituents selected from the group consisting of: —OH, —OR, ═O, ═S, ═N—OH, —O₂CR, —SH, —SR, —SOCR, —NH₂, —NHR, —NR₂, —NR₃+, —NHCOR, —I, —Br, —Cl, —F, —CN, —CO₂H, —CO₂R, —CHO, —COR, —CONH₂, —CONHR, NRCOR, —CONR₂, —COSH, —COSR, —CSOR, NO₂, —OSO₃H, —SO₃H, —SOR and —SO₂R; and benzyl, wherein a phenyl ring of the benzyl is optionally substituted with one or more substituents selected from the group consisting of: R, —OH, —OR, —O₂CR, —SH, —SR, —SOCR, —NH₂, —NHR, —NHR₂, —NHCOR, —I, —Br, —Cl, —F, —CN, —CO₂H, —CO₂R, —CHO, —COR, —CONH₂, —CONHR, —CONR₂, —COSH, —COSR, —NO₂, —SO₃H and —SO₂R; providing neither of R₁ and R₂ is an acyl or thioacyl residue forming an amide with N¹;
Y is a linear, branched, or cyclic, saturated, or unsaturated alkyl chain containing one to ten carbons optionally substituted with one or more substituents selected from the group consisting of: epoxide, —OH, —OR, ═O, ═S, ═N—OH, —O₂CR, —SH, SR, —I, —Br, —Cl, —F, —CN, O₂R, —CHO, —COR, —CONH₂, —CONHR, NRCOR, —CONR₂, NO₂, —SOR and —SO₂R; wherein one or more CH₂ groups if present in the alkyl chain, is optionally replaced by O or S;
R is a linear, branched, or cyclic one to ten carbon saturated, or unsaturated alkyl group optionally substituted with one or more substituents selected from the group consisting of: epoxide, —OH, —OR′, ═O, ═S, ═N—OH, —O₂CR′, —SH, —SR′, —SOCR′, —OSO₃H, —NH₂, —NHR′, —NHR′₂, —NR₃+, —NHCOR′, —NR′COR′, —I, —Br, —Cl, —F, —CN, —CO₂H, CO₂R′, —CHO, —COR′, CONH₂, —CONHR′, —CONR′₂, —COSH, COSR′, —NO₂, —SO₃H, —SOR′ and —SO₂R′, wherein R′ is a linear, branched, or cyclic one to ten carbon, saturated or unsaturated alkyl group optionally substituted with —NH₂;
and wherein formula II is: 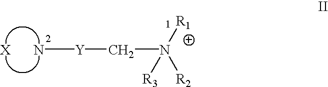
wherein;
X is a saturated, or unsaturated linear, branched, or partially cyclized alkyl chain of between eleven and thirty carbons optionally substituted with one or more substituents selected from the group consisting of: epoxide, ketone, thiocarbonyl, oxime, —OH, —OR, —O₂CR, —SH, —SR, —SOCR, —NH₂, —NHR, —NR₂, —NR₃+, —NHCOR, —I, —Br, —Cl, —F, —CN, —CO₂H, —CO₂R, —CHO, —COR, CONH₂, CONHR, NRCOR, —CONR₂, —COSR, —NO₂, —OSO₃H, —SO₃H, —SOR and —SO₂R; wherein one or more CH₂ groups in the alkyl chain if present, is optionally replaced by a moiety selected from the group consisting of: O, S, NH and NR; and wherein one or more C or CH groups in the alkyl chain if present, is optionally replaced with NH or NR;
R₁, R₂, and R₃ are independently selected from the group consisting of: methyl; a linear, branched, or cyclic, saturated, or unsaturated alkyl group containing one to ten carbons optionally substituted with one or more substituents selected from the group consisting of: —OH, —OR, ═O, ═S, ═N—OH, —O₂CR, —SH, —SR, —SOCR, —NH₂, —NHR, —NR₂, —NR₃+, —NHCOR, —I, —Br, —Cl, —F, —CN, —CO₂H, —CO₂R, —CHO, —COR, —CONH₂, —CONHR, NRCOR, —CONR₂, —COSH, —COSR, —CSOR, NO₂, —OSO₃H, —SO₃H, —SOR and —SO₂R; and benzyl, wherein a phenyl ring of the benzyl is optionally substituted with one or more substituents selected from the group consisting of: R, —OH, OR, —O₂CR, —SH, —SR, —SOCR, —NH₂, —NHR, —NHR₂, —NHCOR, —I, —Br, —Cl, —F, —CN, —CO₂H, —CO₂R, —CHO, —COR, —CONH₂, —CONHR, —CONR₂, —COSH, —COSR, —NO₂, SO₃H, —SOR and —SO₂R; providing none of R₁, R₂, and R₃ is an acyl or thioacyl residue forming an amide with N¹;
Y is a linear, branched, or cyclic, saturated, or unsaturated alkyl chain containing one to ten carbons optionally substituted with one or more substituents selected from the group consisting of: epoxide —OH, —OR, ═O, ═S, ═N—OH, —O₂CR, —SH, —SR, —I, —Br, —Cl, —F, —CN, —CO₂R, —CHO, —COR, —CONH₂, —CONHR, NRCOR, —CONR₂, NO₂, —SOR and —SO₂R; wherein one or more CH₂ groups if present in the alkyl chain, is optionally replaced by O or S; and,
R is a linear, branched, or cyclic one to ten carbon saturated, or unsaturated all group optionally substituted with one or more substituents selected from the group consisting of: epoxide, —OH, —OR′; ═O, ═S, ═N—OH, —O₂CR′, —SH, —SR′, —SOCR′, —OSO₃H, —NH₂, —NHR′, —NHR′₂, —NR′₃+, —NHCOR′, NR′COR′, —I, —Br, —Cl, —F, —CN, —CO₂H, —CO₂R′; —CHO, —COR′, —CONH₂, —CONHR′, —CONR₂, —COSH, —COSR′, —NO₂, —SO₃H, —SOR′ and —SO₂R′, wherein R′ is a linear, branched, or cyclic one to ten carbon, saturated, or unsaturated alkyl group optionally substituted with —NH₂.

2. The composition of claim 1, wherein Y is optionally substituted (CH₂)ₙ in which n is 1-5.

3. The composition of claim 1, wherein X is a saturated linear or branched alkyl chain of 11-16 carbon atoms, optionally substituted with R.

4. The composition of claim 1, wherein X is an unsaturated linear or branched alkyl chain of 1-16 carbon atoms, optionally substituted with R.

5. The composition of claim 1, wherein X is a fully unsaturated and partially cyclized linear alkyl chain of 11-16 carbon atoms, optionally substituted with R.

6. The composition of claim 1, wherein the compound is of formula I in which one or both R₁ and R₂ is a linear or branched alkyl group optionally substituted by a substituent selected from the group consisting of, NH₂, —NHR, —NR₂, —NR₃+ and —NHCOR.

7. The composition of claim 1, wherein the compound is of formula I in which one or both R₁ and R₂ is selected from the group consisting of: hydrogen; methyl; and a linear or branched alkyl group, optionally substituted with a substituent selected from the group consisting of: —OH, —OR, and ═O.

8. The composition of claim 1, wherein the compound is of formula I in which one or both R₁ and R₂ is a linear or branched C₂ to C₆ alkyl group, optionally substituted with a substituent selected from the group consisting of: NH₂, —NHR, —NR₂, —NR₃+ and —NHCOR.

9. The composition of claim 1, wherein the compound is of formula I in which one or both R₁ and R₂ is selected from the group consisting of: hydrogen; methyl; and a linear or branched C₂ to C₆ alkyl group, optionally substituted with a substituent selected from the group consisting of: —OH, —OR, and ═O.

10. The composition of claim 1, wherein the compound is of formula II in which one or more of R₁, R₂, and R₃ is a linear or branched alkyl group, optionally substituted with a substituent selected from the group consisting of: NH₂, —NHR, —NR₂, —NR₃+ and —NHCOR.

11. The composition of claim 1, wherein the compound is of formula II in which one or more of R₁, R₂, and R₃ is selected from the group consisting of: methyl; and a linear or branched alkyl group optionally substituted with a substituent selected from the group consisting of: —OH, —OR, and ═O.

12. The composition of claim 1, wherein the compound is of formula II in which one or more of R₁, R₂, and R₃ is a linen or branched C₂ to C₆ alkyl group, optionally substituted with a substituent selected from the group consisting of: NH₂, —NHR, —NR₂, —NR₃+ and —NHCOR.

13. The composition of claim 1, wherein the compound is of formula II in which one or more of R₁, R₂, and R₃ is selected from the group consisting of: methyl; and a linear or branched C₂ to C₆ alkyl group, optionally substituted with a substituent selected from the group consisting of: —OH, —OR, and ═O.

14. The composition of claim 1, wherein the compound is of formula I in which:
(a) Y is (CH₂)ₙ and n is 1, 2, or 3;
(b) X is a saturated or unsaturated linear alkyl chain of 11-15 carbon atoms, optionally substituted with a C₁-C₆ linear or branched alkyl group; or, a fully unsaturated and partially cyclized linear alkyl chain of 11-16 carbon atoms;
(c) one of R₁ and R₂ is selected from the group consisting of; H, methyl, and a linear or branched C₂-C₆ alkyl group; and,
(d) another of R₁ and R₂ is a linear or branched C₂-C₆ alkyl group optionally substituted with a substituent selected from the group consisting of: NH₂, —NHR, —NR₃+, and —NHCOR, wherein R is a linear or branched C₁-C₆ saturated or unsaturated alkyl group.

15. The composition of claim 1, wherein the compound is of formula II in which:
(a) Y is (CH₂)ₙ and n is 1, 2, or 3;
(b) X is a saturated or unsaturated linear alkyl chain of 11-15 carbon atoms, optionally substituted with , with a C₁-C₆ linear or branched alkyl group; or, a fully unsaturated and partially cyclized linear alkyl chain of 11-16 carbon atoms;
(e) one or two of R₁, R, and R₃ is methyl, or a linear or branched C₂-₆ alkyl group; and,
(d) another of R₁, R, and R₃ is a linear or branched C₂-C₆ alkyl group optionally substituted with a substituent selected from the group consisting of: NH₂, —NHR, NR₃+ and —NHCOR, wherein R is a linear or branched C₁-C₆ saturated or unsaturated alkyl group.

16. The composition of claim 1, wherein the cellular invasion or angiogenesis inhibiting compound or compounds in the composition do not consist exclusively of Motuporamine A, B, or C, or mixtures thereof.

17. The composition of claim 1, wherein a compound in the composition has the structure: 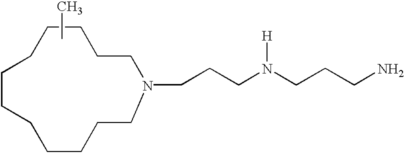
wherein the CH₃ group is joined at one of C-12, C-13, C-14 and C-15.

18. The composition of claim 1, wherein a compound in the composition is selected from the group consisting of: Motuporamine A, B, and C.

19. The composition of claim 1, wherein a compound in the composition is Motuporamine D.

20. The composition of claim 1, wherein a compound in the composition is Dihydromotuporamine C.

21. The composition of claim 1, wherein a compound of the structure: 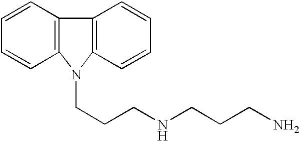
is present in the composition.

22. A cellular invasion or angiogenesis inhibiting compound of formula I or II in substantially purified form or a pharmaceutically acceptable salt thereof, whereby formula I is: 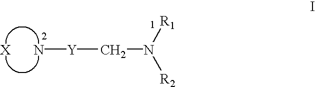
wherein:
X is a saturated, or unsaturated linear, branched, or partially cyclized alkyl chain of between eleven and thirty carbons optionally substituted with one or more substituents selected from the group consisting of: epoxide, ketone, thiocarbonyl, oxime, —OH, —OR, —O₂CR, —SH, —SR, —SOCR, —NH₂, —NHR, —NR₂, —NR³+, —NHCOR, —I, —Br, —Cl, —P, —CN, —CO₂H, —CO₂R, —CHO, —COR, —CONH₂, —CONHR, NRCOR, —CONR₂, —COSR, —NO₂, —OSO₃H, —SO₃H, —SOR and —SO₂R; wherein one or more CH₂ groups if present in the alkyl chain, is optionally replaced by a moiety selected from the group consisting of: O, S, NH and NR; and wherein one or more C and CH groups if present in the alkyl chain, is optionally replaced with NH or NR;
R₁ and R₂ are independently selected from the group consisting of, hydrogen; methyl; a linear, branched, or cyclic saturated, or unsaturated alkyl group containing one to ten carbons optionally substituted with one or more substituents selected from the group consisting of: —OH, —OR, ═O, ═S, ═N—OH, —O₂CR, —SH, —SR, —SOCR, —NR₂, —NHR, —NR₂, —NR₃+, —NHCOR, —I, —Br, —Cl, —F, —CN, —CO₂H, —CO₂R, —CHO, —COR, —CONH₂, —CONHR, NRCOR, —CONR₂, —COSH, —COSR, —CSOR, NO₂, OSO₃H, —SO₃H, —SOR and —SO₂R; and benzyl, wherein a phenyl ring of the benzyl is optionally substituted with one or more substituents selected from the group consisting of: R, —OH, —OR, —O₂CR, —SH, —SR, —SOCR, —NH₂, —NHR, —NHR₂, —NHCOR, —I, —Br, —Cl, —F, —CN, —CO₂H, —CO₂R, —CHO, —COR —CONH₂, —CONHR, —CONR₂, —COSH, —COSR, —NO₂, —SO₃H and —SO₂R; providing neither of R₁ and R₂ is an acyl or thioacyl residue forming an amide with N¹;
Y is a linear, blanched, or cyclic, saturated, or unsaturated alkyl chain containing one to ten carbons optionally substituted with one or more substituents selected from the group consisting of: epoxide, —OH, —OR, ═O, ═S, —N—OH, —O₂CR, —SH, SR, —I, —Br, —Cl, —F, —CN, —CO₂R, —CHO, —COR, —CONH₂, —CONHR, NRCOR, —CONR₂, NO₂, —SOR and —SO₂R; wherein one or more CH₂ groups if present in the alkyl chain, is optionally replaced by O or S;
R is a linear, branched, or cyclic one to ten carbon saturated, or unsaturated alkyl group optionally substituted with one or more substituents selected from the group consisting of: epoxide, —OH, —OR′, ═O, ═S, ═N—OH, —O₂CR′, —SH, —SR′, —SOCR′, —OSO₃H, —NH₂, —NHR′, —NHR′₂, —NR₃+, —NHCOR′, NH′COR′, —I, —Br, —Cl, —F, —CN, —CO₂H, —CO₂R′, —CHO, —COR′, CONH₂, —CONHR′, —CONR′₂, —COSH, —COSR′, —NO₂, —SO₃H, —SOR′ and —SO₂R′, wherein R′ is a linear, branched, or cyclic one to ten carbon, saturated, or unsaturated alkyl group optionally substituted with —NH₂;
and wherein formula II is: 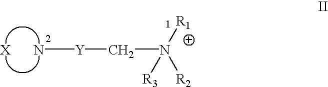
wherein:
X is a saturated, or unsaturated linear, branched, or partially cyclized alkyl chain of between eleven and thirty carbons optionally substituted with one or more substituents selected from the group consisting of: epoxide, ketone, thiocarbonyl, oxime, —OH, —OR, —O₂CR, —SH, —SR, —SOCR, —NH₂, —NHR, —NR₂, —NR₃+, —NHCOR, —I, —Br, —Cl, —F, —CN, —CO₂H, —CO₂R, —CHO, —COR, —CONH₂, —CONHR, NRCOR, —CONR₂, —COSR, —NO₂, —OSO₃H, —SO₃H, —SOR and —SO₂R; wherein one or more CH₂ groups in the alkyl chain if present, is optionally replaced by a moiety selected from the group consisting of: O, S, NH and NR; and wherein one or more C or CH groups in the alkyl chain if present, is optionally replaced with NH or NR;
R₁, R₂, and R₃ are independently selected from the group consisting of: methyl; a linear, branched, or cyclic, saturated, or unsaturated alkyl group containing one to ten carbons optionally substituted with one or more substituents selected from the group consisting of: —OH, —OR, ═O, ═S ═N—OH, —O₂CR, —SH, —SR, —SOCR, —NH₂, —NHR, —NR₂, —NR₃+, —NHCOR, —I, —Br, —Cl, —F, —CN, —CO₂H, —CO₂R, —CHO, —COR, —CONH₂, —CONHR, NRCOR, —CONR₂, —COSH, —COSR, —CSOR, NO₂, —OSO₃H, —SO₃H, —SOR and —SO₂R; and benzyl, wherein a phenyl ring of the benzyl is optionally substituted with one or more substituents selected from the group consisting of: R, —OH, OR, —O₂CR, —SH, —SR, —SOCR, —NH₂, —NHR, —NHR₂, —NHCOR, —I, —Br, —Cl, —F, —CN, —CO₂H, —CO₂R, —CHO, —COR, —CONH₂, —CONHR, —CONR₂, —COSH, —COSR, —NO₂, SO₃H, —SOR and —SO₂R; providing none of R₁, R₂, and R₃ is an acyl or thioacyl residue forming an amide with N¹;
Y is a linear, branched, or cyclic, saturated, or unsaturated alkyl chain containing one to ten carbons optionally substituted with one or more substituents selected from the group consisting of epoxide —OH, —OR, ═O, ═S, ═N—OH, —O₂CR, —SH, —SR, —I, —Br, —Cl, —F, —CN, —CO₂R, —CHO, —COR, —CONH₂, —CONHR, NRCOR, —CONR₂, NO₂, —SOR and —SO₂R; wherein one or more CH₂ groups if present in the alkyl chain, is optionally replaced by O or S; and,
R is a linear, branched, or cyclic one to ten canton saturated, or unsaturated alkyl group optionally substituted with one or more substituents selected from the group consisting of: epoxide, —OH, —OR′, ═O, ═S, ═N—OH, —O₂CR′, —SH, —SR′, —SOCR′, —OSO₃H, —NH₂, —NHR′, —NHR′₂, —NR′₃+, —NHCOR′, NR′COR′, —I, —Br, —Cl, —F, —CN, —CO₂H, —CO₂R′, —CHO, —COR′, —CONH₂, —CONHR′, —CONR′₂, —COSH, —COSR′, —NO₂, —SO₃H, —SOR′ and —SO₂R′, wherein R′ is a linear, branched, or cyclic one to ten carbon, saturated, or unsaturated alkyl group optionally substituted with —NH₂;
and providing that the compound is not Motuporamine A, B, or C.

23. The compound of claim 22, wherein Y is optionally substituted (CH₂)ₙ in which n is 1-5.

24. The compound of claim 22, wherein X is a saturated linear or branched alkyl chain of 11-16 carbon atoms, optionally substituted with R.

25. The compound of claim 22, wherein X is an unsaturated linear or branched alkyl chain of 11-16 carbon atoms, optionally substituted with R.

26. The compound of claim 22, wherein X is a fully unsaturated and partially cyclized linear alkyl chain of 11-16 carbon atoms, optionally substituted with R.

27. The compound of claim 22, wherein the compound is of formula I in which one or both R₁ and R₂ is a linear or branched alkyl group optionally substituted by a substituent selected from the group consisting of: NH₂, —NHR, —NR₂, —NR₃+ and —NHCOR.

28. The compound of claim 22, wherein the compound is of formula I in which one or both R₁ and R₂ is selected from the group consisting of: hydrogen; methyl; and a linear or branched alkyl group, optionally substituted with a substituent selected from the group consisting of: —OH, —OR, and ═O.

29. The compound of claim 22, wherein the compound is of formula I in which one or both R₁ and R₂ is a linear or branched C₂ to C₆ alkyl group, optionally substituted with a substituent selected from the group consisting of: NH₂, —NHR, —NR₂, —NR₃+ and —NHCOR.

30. The compound of claim 22, wherein the compound is of formula I in which one or both R₁ and R₂ is selected from the group consisting of; hydrogen; methyl; ad a linear or branched C₃ to C₆ alkyl group, optionally substituted with a substituent selected from the group consisting of: —OH, —OR, and ═O.

31. The compound of claim 22, wherein the compound is of formula II in which one or more of R₁, R₂, and R₃ is a linear or branched alkyl group, optionally substituted with a substituent selected from the group consisting of: NH₂, —NHR, —NR₂, —NR₃+ and —NHCOR.

32. The compound of claim 22, wherein the compound is of Formula II in which one or more of R₁, R₂, and R₃ is selected from the grow consisting of: methyl; and a linear or branched alkyl group optionally substituted with a substituent selected from the group consisting of: —OH, —OR, and ═O.

33. The compound of claim 22, wherein the compound is of formula II in which one or more of R₁, R₂, and R₃ is a linear or branched C₂ to C₆ alkyl group, optionally substituted with a substituent selected from the group consisting of: NH₂, —NHR, —NR₂, —NR₃+ and —NHCOR.

34. The compound of claim 22, wherein the compound is of formula II in which one or more of R₁, R₂, and R₃ is selected from the group consisting of: methyl; and a linear or branched C₂ to C₆ alkyl group, optionally substituted with a substituent selected From the group consisting of: —OH, —OR, and ═O.

35. The compound of claim 22, wherein the compound is of formula I in which:
(a) Y is (CH₂)ₙ and n is 1, 2, or 3;
(b) X is a saturated or unsaturated linear alkyl chain of 11-15 carbon atoms, optionally substituted with a C₁-C₆ linear or branched alkyl group; or, a fully unsaturated and partially cyclized linear alkyl chain of 11-16 carbon atoms;
(C) one of R₁ and R₂ is selected from the group consisting of: H, methyl, and a linear or branched C₂-C₆ alkyl group; and,
(d) another of R₁ and R₂ is a linear or branched C₂-C₆ alkyl group optionally substituted with a substituent selected from the group consisting of: NH₂, —NHR, —NR₃+, and —NHCOR, wherein R is a linear or branched C₁-C₆ saturated or unsaturated alkyl group.

36. The compound of claim 22, wherein the compound is of formula II in which:
(a) Y is (CH₂)ₙ and n is 1, 2, or 3;
(b) X is a saturated or unsaturated linear alkyl chain of 11-15 carbon atoms, optionally substituted with R, with a C₁-C₆ linear or branched alkyl group; or, a fully unsaturated and partially cyclized linear alkyl chain of 11-16 carbon atoms;
(c) one or two of R₁, R, and R₃ is methyl, or a linear or branched C₂-C₆ alkyl group; and,
(d) another of R₁, R, and R₃ is a linear or branched C₂-C₆ alkyl group optionally substituted with a substituent selected from the group consisting of: NH₂, —NHR, NR₃+ and —NHCOR, wherein R is a linear or branched C₁-C₆ saturated or unsaturated alkyl group.

37. The compound of claim 22, wherein the compound has the structure: 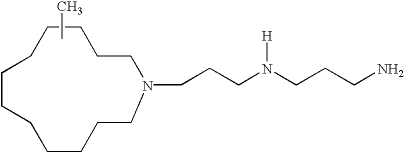
wherein the CH₃ group is joined at one of C12, C-13, C-14 and C-15.

38. The compound of claim 22, wherein the compound is Motuporamine D.

39. The compound of claim 22, wherein the compound is Dihydromotuporamine C.

40. A method for inhibiting cellular invasion or angiogenesis in a patient in need thereof, comprising administering to the patient, an amount of a compound or pharmaceutically acceptable salt thereof effective to inhibit cellular invasion or angiogenesis in a tissue of the patient, the compound being of formula I or II, wherein formula I is: 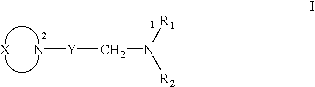
wherein:
X is a saturated, or unsaturated linear, branched, or partially cyclized alkyl chain of between eleven and thirty carbons optionally substituted with one or more substituents selected from the group consisting of: epoxide, ketone, thiocarbonyl, oxime, —OH, —OR, —O₂CR, —SH, —SR, —SOCR, —NH₂, —NHR, —NR₂, —NR³+, —NHCOR, —I, —Br, —Cl, —F, —CN, —CO₂H, —CO₂R, —CHO, —COR, —CONH₂, —CONHR, NRCOR, —CONR₂, —COSR, —NO₂, —OSO₃H, —SO₃H, —SOR and —SO₂R; wherein one or more CH₂ groups if present in the alkyl chain, is optionally replaced by a moiety selected from the group consisting of: O, S, NH and NR; and wherein one or more C and CH groups if present in the alkyl chain, is optionally replaced with NH or NR;
R₁ and R₂ are independently selected from the group consisting of: hydrogen; methyl; a linear, branched, or cyclic saturated, or unsaturated alkyl group containing one to ten carbons optionally substituted with one or more substituents selected from the group consisting of: —OH, —OR, ═O, ═S, ═N—OH, —O₂CR, —SH, —SR, —SOCR, —NH₂, —NHR, —NR₂, —NR₃+, —NHCOR, —I, —Br, —Cl, —F, —CN, —CO₂H, —CO₂R, —CHO, —COR, —CONH₂, —CONHR, NRCOR, CONR₂, —COSH, —COSR, —CSOR, NO₂, —OSO₃H, —SO₃H, —SOR and —SO₂R; and benzyl, wherein a phenyl ring of the benzyl is optionally substituted with one or more substituents selected from the group consisting of: R, —OH, —OR, —O₂CR, —SH, —SR, —SOCR, —NH₂, —NHR, —NHR₂, —NHCOR, —I, —Br, —Cl, —F, —CN, —O₂H, —CO₂R, —CHO, —COR, —CONH₂, —CONHR, —CONR₂, —COSH, —COSR, —NO₂, —SO₃H and —SO₂R; providing neither of R₁ and R₂ is an acyl or thioacyl residue forming an amide with N¹;
Y is a linear, branched, or cyclic, saturated, or unsaturated alkyl chain containing one to ten carbons optionally substituted with one or more substituents selected from the group consisting of: epoxide, —OH, —OR, ═O, ═S, ═N—OH, —O₂CR, —SH, SR, —I, —Br, —Cl, —F, —CN, —CO₂R, —CHO, —COR, —CONH₂, —CONHR, NRCOR, —CONR₂, NO₂, —SOR and —SO₂R; wherein one or more CH₂ groups if present in the alkyl chain, is optionally replaced by O or S;
R is a linear, branched, or cyclic one to ten carbon saturated, or unsaturated alkyl group optionally substituted with one or more substituents selected from the group consisting of: epoxide, —OH, —OR′, ═O, ═S, ═N—OH, —O₂CR′, —SH, —SR′, —SOCR; —OSO₃H, —NH₂, —NHR′, —NHR′₂, —NR₃+, —NHCOR, NR′COR′, —I, —Br, —Cl, —F, —CN, —CO₂H, —CO₂R′, —CHO, —COR′, CONH₂, —CONHR′, —CONR′, —COSH, —COSR′, —NO₂, —SO₃H, —SOR′ and —SO₂R′; wherein R′ is a linear, branched, or cyclic one to ten carbon, saturated, or unsaturated alkyl group optionally substituted with —NH₂;
and wherein formula II is: 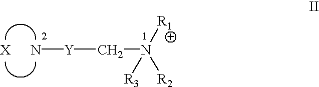
wherein:
X is a saturated, or unsaturated linear, branched, or partially cyclized alkyl chain of between eleven and thirty carbons optionally substituted with one or more substituents selected from the group consisting of: epoxide, ketone, thiocarbonyl, oxide, —OH, —OR, —O₂CR, —SH, —SR, —SOCR, —NH₂, —NHR, —NR₂, —NR₃+, —NHCOR, —I, —Br, —Cl, —F, —CN, —CO₂H, —CO₂R, —CHO, —COR, —CONH₂, —CONHR, NRCOR, —CONR₂, —COSR, —NO₂, —OSO₃H, —SO₃H, —SOR and —SO₂R; wherein one or more CH₂ groups in the alkyl chain if present, is optionally replaced by a moiety selected from the group consisting of O S, NH and NR; and wherein one or more C or CH groups in the alkyl chain if present, is optionally replaced with NH or NR,
R₁, R₂, and R₃ are independently selected from the group consisting of: methyl; a linear, branched, or cyclic, saturated, or unsaturated alkyl group containing one to ten carbons optionally substituted with one or more substituents selected from the group consisting of: —OH, —OR, ═O, ═S, ═N—OH, —O₂CR, —SH, —SR, —SOCR, NH₂, —NHR, —NR₂, —NR₃+, —NHCOR, —I, —Br, —Cl, —F, —CN, —CO₂R, —CO₂R, —CHO, —COR, —CONH₂, —CONHR, NRCOR, —CONR′, —COSH, —COSR, —CSOR, NO₂, —OSO₃H, —SO₃H, —SOR and —SO₂R, and benzyl, wherein a phenyl ring of the benzyl is optionally substituted with one or more substituents selected from the group consisting of: R, —OH, OR, —O₂CR, —SH, —SR, —SOCR, —NH₂, —NHR, —NHR₂, —NHCOR, —I, —Br, —Cl, —F, —CN, —CO₂H, —CO₂R, —CHO, —COR, —CONH₂, CONHR, —CONR₂, —COSH, —COSR, —NO₂, SO₃H, —SOR and —SO₂R; providing none of R₁, R₂, and R₃ is an acyl or thioacyl residue forming an amide with N¹;
Y is a linear, branched, or cyclic, saturated, or unsaturated alkyl chain containing one to ten carbons optionally substituted with one or more substituents selected from the group consisting of: epoxide —OH, —OR, ═O, ═S, ═N—OH, —O₂CR, —SH, —SR, —I, —Br, —Cl, —F, —CN, —CO₂R, —CHO, —COR, —CONR₂, —CONHR, —CONHR₂, NRCOR, —CONR₂, NO₂, —SOR and —SO₂R; wherein one or more CH₂ groups if present in the alkyl chain, is optionally replaced by O or S; and,
R is a linear, branched, or cyclic one to ten carbon saturated, or unsaturated alkyl group optionally substituted with one or more substituents selected from the group consisting of: epoxide, —OH, —OR′, ═O, ═S, ═N—OH, —O₂CR′, —SH, —SR′, —SOCR′; —OSO₃H, —NR₂, —NHR′, —NHR′₂, —NR′₃+, —NHCOR′, NR′COR′, —I, —Br, —Cl, —F, —CN, —CO₂H, —CO₂R′, —CHO, —COR′, —CONH₂, —CONHR′, —CONR′₂, —COSH, —COSR′, —NO₂, —SO₃H, —SOR′ and —SO₂R; wherein R′ is a linear, branched, or cyclic one to ten carbon, saturated, or unsaturated alkyl group optionally substituted wit —NH₂.

41. The method of claim 40, wherein
(a) Y is (CH₂)ₙ and n is 1, 2, or 3;
(b) X is a saturated or unsaturated linear all chain of 11-15 carbon atoms, optionally substituted with a C₁-C₆ linear or branched alkyl group; or, a frilly unsaturated partially cyclized linear alkyl chain of 11-16 carbon atoms;
(c) one of R₁ and R₂ is selected from the group consisting of: H, methyl, and a linear or branched C₂-C₆ alkyl group; and,
(d) another of R₁ and R₂ is a linear or branched C₂-C₆ alkyl group optionally substituted with a substituent selected from the group consisting of: NH₂, —NHR, —NR₃+, and —NHCOR, wherein R is a linear or branched C₁-C₆ saturated or unsaturated alkyl group.

42. The method of claim 40, wherein the compound is of formula II in which:
(a) Y is (CH₂)ₙ and n is 1, 2, or 3;
(b) X is a saturated or unsaturated linear alkyl chum of 11-15 carbon atoms, optionally substituted with R, with a C₁-C₆ linear or branched alkyl group; or, a fully unsaturated and partially cyclized linear alkyl chain of 11-16 carbon atoms;
(c) one or two of R₁, R, and R₃ is methyl, or a linear or branched C₂-C₆ alkyl group; and
(d) another of R₁, R, and R₃ is a linear or branched C₂-C₆ alkyl group optionally substituted with a substituent selected from the group consisting of: NH₂, —NHR, —NHR₃+ and —NHCOR, wherein R is a linear or branched C₁-C₆ saturated or unsaturated alkyl grove.

43. The method of claim 40, wherein a compound in the composition has the structure: 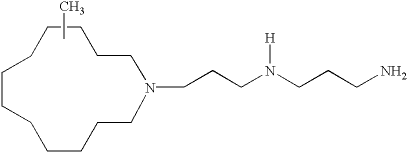
wherein the CH₃group is joined atone of C12, C-13, C-14 and C-15.

44. The method of claim 40, wherein the compound is selected from the group consisting of Motuporamine A, B, and C.

45. The method of claim 40, wherein the compound is Motuporamine D.

46. The method of claim 40, wherein the compound is Dihydromotuporamine C.

47. A method for testing for the presence of an agent that inhibits cellular invasion comprising:
(a) placing cells on a surface of a biological matrix into which said cells are capable of invasion;
(b) treating said cells with an agent to be tested for cellular invasion inhibition activity;
(c) maintaining the cells on the surface of the matrix for a time sufficient for the cells to invade the matrix in the absence of an agent which inhibits cellular invasion;
(d) removing substantially all cells from the surface of the matrix after (c);
(e) transferring the cells removed at (d) to a surface upon which said cells are capable of attachment and proliferation;
(f) maintaining the cells on a surface at (e) for a time sufficient for cellular attachment and proliferation on the surface;
(g) determining a value indicative of a quantity of cells attached to said surface after (f); and,
(h) comparing the value determined at (g) to a control value determined by performing (a), (c), (d), (e), (f) and (g) with the cells in the absence of an agent that inhibits cellular invasion.

48. The method of claim 47, wherein the cells are cancer cells.

49. The method of claim 47, wherein the matrix is a membrane or a gel.

50. The method of claim 49, wherein the matrix comprises Matrigel™.
